(19) United States
(12) Patent Application Publication
(10) Pub. No.: US 2011/0288149 A1
(43) Pub. Date: Nov. 24, 2011

(54) Method of protecting against heart failure

(75) Inventors: Douglas A. Marchuk, Durham, NC (US); Howard A. Rockman, Durham, NC (US); Ferrin C. Wheeler, Durham, NC (US)

(73) Assignee: Duke University

(21) Appl. No.: 13/067,013

(22) Filed: May 2, 2011

Related U.S. Application Data
(63) Continuation-in-part of application No. PCT/US2009/005922, filed on Nov. 2, 2009.
(60) Provisional application No. 61/110,323, filed on Oct. 31, 2008.

(51) Int. Cl. A61K 31/713; C07K 2/00; A61P 9/00; C12Q 1/48; A01K 67/027

(52) U.S. Cl. 514/44 A; 435/15; 800/18; 530/300

(57) ABSTRACT

The present invention relates, in general, to heart failure, and, in particular to a method of reducing the risk of heart failure, particularly in patents with established cardiomyopathy.

This application is a continuation-in-part of International Application No. PCT/US2009/005922, filed Nov. 2, 2009 which claims priority from U.S. Provisional Application No. 61/110,323, filed Oct. 31, 2008, the entire contents of which are hereby incorporated by reference.

This invention was made with government support under Grant Nos. R01 HL083155, R01 HL68963 and 5 F32HL079863 awarded by the National Institutes of Health. The government has certain rights in the invention.

### TECHNICAL FIELD

The present invention relates, in general, to heart failure, and, in particular to a method of reducing the risk of heart failure, particularly in patents with established cardiomyopathy.

### BACKGROUND

Genetic factors contributing to the progression and severity of heart disease have been difficult to identify in large part due to the challenge of standardizing clinical outcomes in human populations. Thus, forward genetic approaches have had limited success in identifying novel therapeutic targets.

The Calsequestrin (CSQ) transgenic mouse model of cardiomyopathy (Jones et al, J. Clin. Invest. 101:1385-1393 (1998), Cho et al, J. Biol. Chem. 274:22251-22256 (1999)) exhibits wide variation in phenotypic progression dependent on genetic background (Suzuki et al, Circulation 105:1824-1829 (2002), Le Corvoisier et al, Hum. Mol. Genet. 12:3097-3107 (2003)). Quantitative trait locus (QTL) mapping using a CSQ transgenic sensitizer has yielded seven heart failure modifier (Hrtfm) loci that modify disease progression and outcome (Suzuki et al, Circulation 105:1824-1829 (2002), Le Corvoisier et al, Hum. Mol. Genet. 12:3097-3107 (2003), Wheeler et al, Mamm. Genome 16:414-423 (2005)). Hrtfm2, mapped in two different crosses (Suzuki et al, Circulation 105:1824-1829 (2002), Wheeler et al, Mamm. Genome 16:414-423 (2005)), accounts for 28-30% of the phenotypic variance in survival, and 22-42% of the phenotypic variance in heart function.

The present invention results, at least in part, from the identification of Tnni3k (cardiac Troponin I-interacting kinase) as the gene underlying Hrtfm2.

### SUMMARY OF THE INVENTION

The present invention relates generally to heart failure. More specifically, the invention relates to methods of protecting against and/or reducing the risk of heart failure in patients with cardiomyopathy. The invention also relates to methods of identifying agents suitable for use in therapeutic strategies designed to protect against heart failure, particularly in patients with established cardiomyopathy.

This invention was made with government support under Grant Nos. RO1 HL083155, RO1 HL68963 and 5 F32HL079863 awarded by the National Institutes of Health. The government has certain rights in the invention.

Objects and advantages of the present invention will be clear from the description that follows.

### BRIEF DESCRIPTION OF THE DRAWINGS

FIGS. 1A-1C. Tnni3k mRNA and protein expression varies significantly between mouse strains. (A) Affymetrix microarray analysis identified only one gene on murine chromosome 3 with a significant expression change between B6, AKR and DBA. Two genes flanking Tnni3k (Cryz and Lrrc44) that are expressed at similar levels in all strains are shown, as well as two control genes, Actb (β-actin) and Gapdh. (B) qRT-PCR confirms expression differences identified by microarray analysis. TaqMan qRT-PCR of Tnni3k from 5 wild-type mouse hearts from each strain confirms that transcript levels are higher (approximately 25-fold) in B6 and AKR compared to DBA (**p>0.0001 and *p>0.001). Three hearts from the Hrtfm2 congenic line harboring AKR alleles at the Tnni3k locus on a DBA genetic background (DBA.AKR-Hrtfm2) shows transcript levels similar to B6 and AKR hearts, which is significantly higher than observed in DBA hearts (**p>0.0001). Actb served as an endogenous control. Error bars indicate standard error of the mean (SEM). (C) Western blot analysis shows that three strains that share the DBA haplotype at Tnni3k show no detectable Tnni3k protein, while three strains with the B6 haplotype show moderate to high expression. The DBA.AKR-Hrtfm2 congenic mouse shows high expression as predicted based on RNA expression. Receptor tyrosine kinase Tek which shows moderate expression in the heart (http://symatlas.gnf.org/SymAtlas/) was used as a protein loading control (Santa Cruz Biotechnology, Santa Cruz, Calif.).

FIG. 2. Coding and representative non-coding polymorphic SNPs from the Tnni3k genomic region show two distinct haplotype groups. The two SNP haplotypes correlate with Tnni3k transcript levels. Group 1 (DBA, C3H, and Balb/c) shows low levels of Tnni3k while group 2 (B6, AKR, and 129Sv) shows high levels of Tnni3k.

FIG. 3. Western blot analysis of polyclonal antibody raised against a C-terminal mouse Tnni3k peptide. Control blot showing lysates from 293T cells transiently transfected with a mouse Tnni3k expression vector or an empty vector control. Tnni3k protein is visible in the positive control lysate at a size of approximately 90 kDa, as predicted. Also shown are heart lysates from DBA, AKR and B6 mice. DBA does not show Tnni3k protein while AKR and B6 show robust protein expression.

FIGS. 4A-4D. Aberrant splicing of Tnni3k in hearts from DBA mice. (A) Sequencing chromatogram shows the exon 19-20 boundary in Tnni3k cDNA from B6 and DBA hearts. The dashed line shows the first base of the 4 nucleotide cDNA insertion (GTTT) derived from intron 19. The small proportion of properly spliced transcript in DBA can be seen as overlapping sequence after the dashed line. (B) Sequence of exon 19 and 20 with flanking intronic sequence with amino acid translation for both B6 (1^{st} site/normal) and DBA (2^{nd} site/aberrant). The 4 nucleotide GTTT insertion is shown in bold. (C) Fluorescent fragment analysis (GeneMapper, Applied Biosystems) was used to determine the fraction of aberrant splicing in DBA. Almost 70% of total message in DBA was mis-spliced, while no aberrant splicing was observed in B6 and AKR. (D) Weight matrix scores of the different splice donor sites were calculated using a simple additive mathematical model (Staden, Nucleic Acids Res. 12:505-519 (1984), Burset et al, Nucleic Acids Res. 28:4364-4375 (2000)). Calculated strengths of the various donor sites are shown.

FIGS. 5A and 5B. The sequence at rs57952686 is responsible for aberrant Tnni3k splicing. An in vitro system was used to test the role of the intron 19 SNP (rs57952686) in aberrant splicing between exons 19 and 20 and in DBA compared to B6. (A) Schematic representation of the Tnni3k exon 18-20 in vitro splicing construct used to test aberrant splicing. Genomic fragments (4 kb) from DBA and B6 including exons 18, 19 and 20 were amplified and cloned into the pSPL3 splicing vector. Additionally, site-directed mutagenesis was used to alter the sequence at rs57952686 in both constructs. Splicing constructs were transfected into 293T cells and RNA was harvested after 48 hours. (B) Analysis of Tnni3k splicing reveals aberrant splicing of the in vitro DBA construct closely resembles splicing in wild-type DBA hearts but the aberrant transcript is absent with the B6 in vitro construct. When the critical nucleotide at the +9 position in intron 19 is exchanged between the constructs, the splicing pattern follows the sequence at the SNP, demonstrating that the sequence at rs57952686 is responsible for the splicing defect.

FIGS. 6A and 6B. Nonsense mediated decay is responsible for reduced Tnni3k transcript levels. HL-1 cardiomyocytes were treated with emetine or cycloheximide to block NMD. RNA was isolated from cells 24 hours after treatment. Fluorescent RT-PCR fragment analysis was used to measure the ratio of aberrant to wild type transcripts, and qRT-PCR was used to determine Tnni3k message levels relative to actb. Cells that were mock treated acted as a control. (A) Either emetine or cycloheximide treatment preferentially increases levels of the aberrantly-spliced message relative to the normally-spliced message (*p>0.01). (B) Either emetine or cycloheximide treatment increased the total levels of Tnni3k message approximately 16-fold above mock-treated cells (**p>0.001).

FIGS. 7A and 7B. The cross between the congenic line with the Hrtfm2 locus from the AKR line shows decreased cardiac function when crossed to the CSQ transgenic sensitizer line (C+), in comparison with DBA crossed to the transgenic sensitizer. Left ventricular diastolic and systolic diameters are increased in the congenic mice in comparison to DBA mice. This results in a reduced fractional shortening in the congenic lines. The DBA line expresses no detectable Tnni3k protein, whereas the congenic line expresses approximately ½ the levels seen in AKR. These data show that natural levels of mouse Tnni3k expression result in poor cardiac function in comparison to a strain that expresses no detectable protein.

FIG. 8. TNNI3K expression at moderate or high leads to premature death in the CSQ transgenic model of cardiomyopathy. A Kaplan-Meier survival graph shows the outcomes of different genotypic groups resulting from a cross between TNNI3K (T) and CSQ (C) transgenic animals, and a cross between the congenic Hrtfm2 line (described in FIG. 2), and the CSQ (C) transgenic line—resulting in only one copy of the Hrtfm2 locus from AKR (½ congenic). For the cross with the transgenic line, survival is severely decreased for double positive transgenics (T+/C+) to an average of 17 days with a range from 15 to 21 days. Nearly all mice with other genotypes, including both single positives (T+/C−, T−/C+) survived well past the end-point of 150 days. Survival of T+/C+ compared to the three other groups was significantly decreased (p<0.00001). For the cross of the congenic animal containing the AKR genomic segment of Hrtfm2 and The CSQ transgenic, the mice also shows reduced survival relative to controls. The expression level of Tnni3k in these mice is ½ that of B6 or AKR, and approximately 5-20 fold less than the Tnni3k transgenics. The number of animals in each group is as follows: T+/C+, n=12; T+/C−, n=18; T−/C+, n=14; T−/C−, n=18, ½ congenic/C+, n=8.

FIGS. 9A and 9B. Western blot analysis of polyclonal antibody raised against a C-terminal human TNNI3K peptide. (A) Control blot showing a lysate from 293T cells transiently transfected with a human TNNI3K expression vector or an empty vector control. TNNI3K protein is visible only in the TNNI3K lysate at a size of approximately 90 kDa, as predicted from the protein sequence. (B) Western blot with heart lysates from several TNNI3K transgenic mice. Animals from three lines tested positive for the transgene by genotyping (lines 9, 23 and 26). Mice from three generations of line 9 and two generations of line 26 that tested positive for transgenic TNNI3K protein by Western blot are shown. Heart lysates were examined from each generation to ensure continued expression of transgenic protein. SYBR green qRT-PCR analysis of transgenic transcripts showed that levels of TNNI3K transgene expression in TNNI3K transgenic mice ranged from 5-20-fold higher than endogenous Tnni3k measured in B6 heart RNA.

FIGS. 10A and 10B. TNN13K expression leads to severely impaired systolic function in the CSQ transgenic model of cardiomyopathy. M-mode echocardiograms were performed on 14-day old mice from a cross between TNNI3K and CSQ transgenic animals. (A) Representative echocardiograms show that the double positive transgenic mice display severe left ventricle systolic dysfunction and chamber dilation. As expected at this early stage in disease progression, the TNNI3K-/CSQ+ animals shows only a low level of dilation, while the TNNI3K+/CSQ− and the TNNI3K−/CSQ− animals exhibit normal heart function. (B) Table of echocardiographic data from mice with 4 possible genotypes. LVEDd, LVEDs, heart rate, fractional shortening (FS) and mVCFc are shown. Only two double transgenic mice survived the conscious echocardiography at day 14; three others died during the procedure. Individual data is shown separately for the two that survived the procedure. Data is represented by mean±S.D for T−/C−, T+/C− and T−/C+ groups.

FIGS. 11A and 11B. TNNI3K expression leads to systolic dysfunction in a surgically-induced model of cardiomyopathy. Echocardiography was performed prior to transverse aortic constriction (TAC) and at 4- and 8-weeks post TAC surgery. LVEDs (A) and FS (B) were compared between TNNI3K+ mice (n=11) and TNNI3K− littermates (n=13) at 4 and 8 weeks post-TAC. LVEDs were significantly higher in TNNI3K+ mice at 4 and 8 weeks, but were not statistically different prior to surgery. Similarly, fractional shortening was significantly decreased in TNNI3K+ mice at both 4 and 8 weeks following surgery. Error bars represent the standard error of the mean (SEM).

FIG. 12. Amino acid sequence of human Tnni3k and nucleic acid sequence encoding the protein.

FIG. 13. Co-immunostaining was performed on TNNI3K transgenic mouse heart sections using antibodies against TNNI3K (red) and other sarcomeric proteins. TNNI3K shows a reciprocal staining pattern with Myosin (green). TNNI3K staining partially overlaps with F-Actin (Phalloidin, green), and exclusively co-localizes with sarcomere Z-disc protein Desmin (green) in longitudinal sections. In cross-section, TNNI3K localizes inside Desmin ring structures. Each bar represent 5 μm.

FIG. 14. Co-immunostaining was performed on heart sections from C57BL/6J and DBA/2J inbred mice using antibodies against mouse TNNI3K (red) and Desmin (green). Consistent with previous qRT-PCR and western blot result (Wheeler et al, PLoS Genet. September; 5(9):e1000647 (2009). Epub 2009 Sep. 18), TNNI3K Z-disc expression pattern is only detected in C57BL/2J, but not in DBA/2J mouse. TNNI3K is also detected around nucleus (arrow heads).

FIGS. 15A and 15B. TNNI3K interacts with cardiac α-actin (ACTC1) in cultured cells. (FIG. 15A) TNNI3K co-localizes with actin filaments in cultured cells. Cos-7 cells and HL-1 cells were transfected with Flag-tagged hTNNI3K. Transfected cells were stained with anti-Flag (green) to visualize TNNI3K and phalloidin (red) to visualize actin filaments. Flag-TNNI3K co-localizes with actin filaments. The same pattern was seen when TNNI3K was co-transfected with HA-tagged cardiac alpha actin (hACTC1) (100× magnification, * indicates nucleus). (FIG. 15B) TNNI3K co-immunoprecipitates with actin. 293T cells were co-transfected with Flag-hTNNI3K and HA-hACTC1. Lysates were immunoprecipitated with an anti-Flag antibody to pull-down hTNNI3K, and HA-hACTC1 (42 kDa) was detected in the pellet using an anti-HA antibody. In a reciprocal experiment, lysates were immunoprecipitated with an anti-HA antibody to pull-down HA-hACTC1, and Flag-hTNNI3K (93 kDa) was detected in the pellet using an anti-Flag antibody. Single transfections with Flag-hTNNI3K or HA-hACTC1 were performed as controls.

FIGS. 16A-16B. TNNI3K exhibits a strain-specific, striated expression pattern only in heart tissue. (FIG. 16A) Heart sections from C57BL/6J and DBA/2J inbred mice were immunostained using antisera against mouse TNNI3K (red) and desmin (green). TNNI3K shows a striated expression pattern in C57BL/6J hearts that is absent from DBA/2J hearts. In C57BL/6J hearts TNNI3K is also detected around the nucleus (arrows). (FIG. 16B) TNNI3K is not expressed in skeletal muscle. In a western blot using antiserum against mouse TNNI3K, TNNI3K was detected in heart lysate from C57BL/6J mice but not in lysate from DBA/2J mouse hearts, or in skeletal muscle lysates from either strain. Anti-alpha tubulin was used as a loading control. Each bar represents 10 μm.

FIGS. 17A-17L. TNNI3K localizes to the sarcomeric Z disc in cardiomyocytes, and its kinase activity is not required for its localization. TNNI3K localizes to the sarcomeric Z disc. C57BL/6J mouse heart sections were co-immunostained with antisera against mouse TNNI3K (red, FIGS. 17A, 17D, 17G and 17J) and other sarcomeric proteins (green, FIGS. 17B, 17E, 17H and 17K). In longitudinal sections of sarcomeres, TNNI3K shows a reciprocal staining pattern with myosin (FIGS. 17B and 17C), partially overlaps with F-actin (phalloidin, FIGS. 17E and 17F), and co-localizes with desmin (FIGS. 17H and 17I), the intermediate filaments surrounding the Z disc. In cross-section, TNNI3K localizes inside the desmin ring structures (FIGS. 17J-17L). Each bar represents 5 μm.

FIGS. 18A-18C. TNNI3K interacts with the Z disc protein myotilin. (FIG. 18A) Co-localization of Flag-hTNNI3K and HA-hmyotilin in transfected cells. Cos-7 cells and HL-1 cells were transfected with Flag-hTNNI3K and HA-hmyotilin. Immunostaining with anti-Flag (green) and anti-HA (red) antisera shows the co-localization of Flag-hTNNI3K and HA-hmyotilin in transfected cells. (100× magnification). (FIG. 18B) Co-immunoprecipitation of TNNI3K and myotilin in vitro. 293T cells were co-transfected with Flag-hTNNI3K and HA-hmyotilin. Lysates were immunoprecipitated with an anti-Flag antibody to pull-down hTNNI3K, and HA-hmyotilin (55 kDa) was detected in the pellet using an anti-HA antibody. Lysates were also immunoprecipitated with an anti-HA antibody to pull-down HA-hmyotilin, and Flag-hTNNI3K was detected in the pellet using an anti-Flag antibody. Single transfections with Flag-hTNNI3K or HA-hmyotilin were used as controls. (FIG. 18C) Co-immunoprecipitation of TNNI3K and myotilin in vivo. Heart lysates from TNNI3K^{tg} mice were immunoprecipitated using an anti-myotilin antibody or normal rabbit serum as a control. TNNI3K (93 kDa) was detected in the pellets by an immunoblot with an anti-TNNI3K antibody.

FIG. 19. Mapping the TNNI3K actin/myotilin filament association domain. Full-length and truncated Flag-hTNNI3K constructs were transfected into COS-7 cells alone (the first left panel) or co-transfected with HA-myotilin (the three right panels). Their intracellular staining pattern was determined by immunostaining with anti-Flag antibody (green) or anti-HA (red) (100× magnification). Schematic representations of the truncated Flag-hTNNI3K proteins are shown on the left. The staining of both wild type Flag-TNNI3K and Flag-TNKD show the characteristic cytoskeletal staining pattern. By contrast, the isolated ankyrin repeat domain (ANKR) and the kinase domain, with or without the Ser-rich domain, (KinaseDM or ΔANKR) lose this characteristic pattern and instead show diffuse staining throughout the cytoplasm. Deletion of only the serine-rich C-terminal tail (ΔSer) shows reduced cytoskeletal staining with an increase in the diffuse cytoplasmic localization. These data suggest that the complete interaction of TNNI3K with cytoskeletal partners actin and myotilin requires the full domain structure of the protein, but not its kinase activity.

### DETAILED DESCRIPTION OF THE INVENTION

The present invention results, at least in part, from studies demonstrating that levels of Tnni3k are a major determinant of the rate of heart disease progression in mouse models of cardiomyopathy (see Examples below). The studies further demonstrate that the kinase activity of Tnni3k is required for modification of disease progression. The data provided in the Examples that follow indicate that Tnni3k is a sarcomeric Z disc kinase that mediates cytoplasmic signaling to sarcomeric structural proteins to modulate cardiac response to stress. The invention provides methods for identifying compounds that can be used to inhibit the effects of Tnni3k in vivo, including the induction by Tnni3k of premature heart failure in patients with cardiomyopathy. The invention also relates to compounds so identified and to methods of using same to protect against, or reduce the risk of, heart failure in patients with cardiomyopathy.

In one embodiment, the present invention relates to methods of screening compounds for their ability to bind Tnni3k and thereby to function, potentially, as Tnni3k antagonists. Tnni3k includes two recognizable protein motifs: a series of ankyrin repeats in the amino terminus and a tyrosine kinase domain in the carboxy-terminus. The entire Tnni3k molecule can be used in the present screening methods (assays) or a fragment thereof can be used, for example, the tyrosine kinase domain, as can a fusion protein comprising Tnni3k or the fragment thereof.

Binding assays of this embodiment invention include cell-free assays in which Tnni3k or fragment thereof (or fusion protein containing same) is incubated with a test compound (proteinaceous or non-proteinaceous) which, advantageously, bears a detectable label (e.g., a radioactive or fluorescent label). Following incubation, the Tnni3k or fragment thereof (or fusion protein) bound to test compound can be separated from unbound test compound using any of a variety of techniques (for example, Tnni3k (or fragment thereof or fusion protein) can be bound to a solid support (e.g., a plate or a column) and washed free of unbound test compound). The amount of test compound bound to Tnni3k or fragment thereof (or fusion protein) can then be then determined using a technique appropriate for detecting the label used (e.g., liquid scintillation counting and gamma counting in the case of a radiolabelled test compound or by fluorometric analysis). A test compound that binds to Tnni3k (or fragment thereof or fusion) is a candidate inhibitor of Tnni3k activity (e.g., kinase activity).

Binding assays of this embodiment can also take the form of cell-free competition binding assays. In such an assay, Tnni3k or fragment thereof, or fusion protein containing same, can be incubated with a compound known to interact with (e.g., bind to) Tnni3k (e.g., cardiac Troponin I (cTnI) or myelin basic protein (MBP)), which known compound, advantageously, bears a detectable label (e.g., a radioactive or fluorescent label). A test compound (proteinaceous or non-proteinaceous) is added to the reaction and assayed for its ability to compete with the known (labeled) compound for binding to Tnni3k or fragment thereof (or fusion protein). Free known (labeled) compound can be separated from bound known compound, and the amount of bound known compound determined to assess the ability of the test compound to compete. This assay can be formatted so as to facilitate screening of large numbers of test compounds by linking Tnni3k or fragment thereof (or fusion protein) to a solid support so that it can be readily washed free of unbound reactants. A plastic support, for example, a plastic plate (e.g., a 96 well dish), is preferred.

Tnni3k suitable for use in the cell-free assays described above can be isolated from natural sources. Tnni3k or fragment thereof (or fusion protein) can be prepared recombinantly or chemically. Tnni3k, or fragment thereof, can be prepared as a fusion protein using, for example, known recombinant techniques. Preferred fusion proteins include a GST (glutathione-S-transferase) moiety, a GFP (green fluorescent protein) moiety (useful for cellular localization studies) or a His tag (useful for affinity purification). The non-Tnni3k moiety can be present in the fusion protein N-terminal or C-terminal to the Tnni3k moiety.

As indicated above, the Tnni3k or fragment thereof, or fusion protein, can be present linked to a solid support, including a plastic or glass plate or bead, a chromatographic resin (e.g., Sepharose), a filter or a membrane. Methods of attachment of proteins to such supports are well known in the art.

The binding assays of the invention also include cell-based assays. Cells suitable for use in such assays include cells that naturally express Tnni3k and cells that have been engineered to express Tnni3k (or fragment thereof or fusion protein comprising same). Advantageously, cells expressing human Tnni3k are used. Examples of suitable cells include cardiac cells (e.g., human cardiac cells (such as cardiomyocytes)).

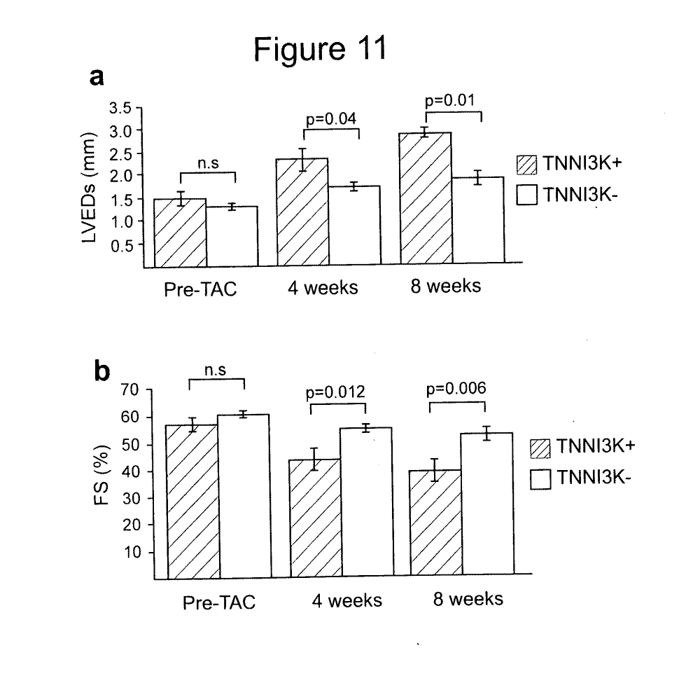

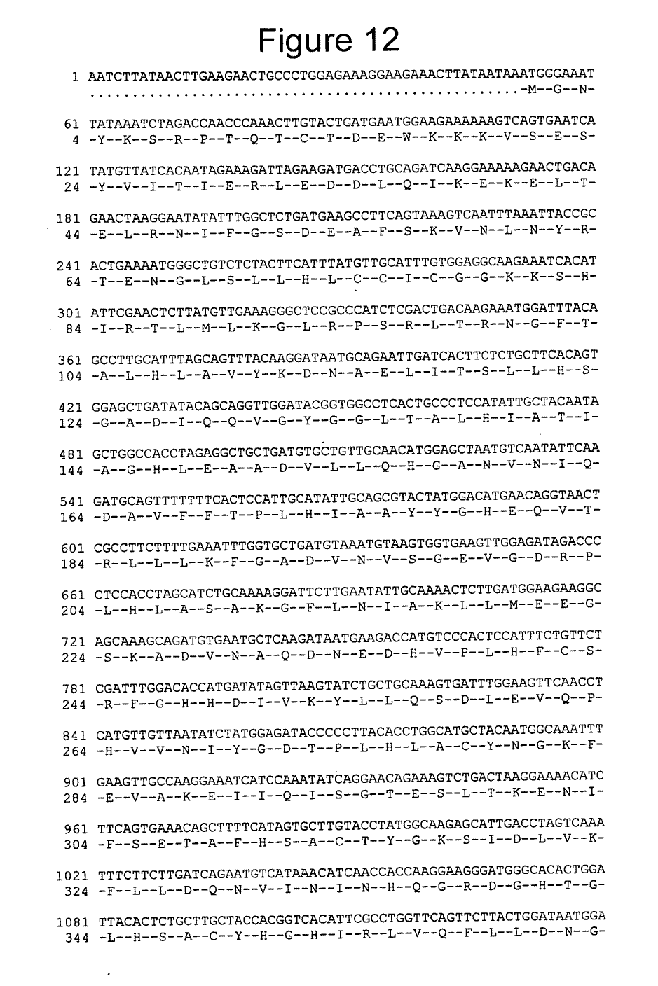

Cells can be engineered to express Tnni3k (advantageously, human Tnni3k or fragment thereof, or fusion protein that includes same) by introducing into a selected host cell an expression construct comprising a sequence encoding Tnni3k (e.g., the encoding sequence shown in FIG. 11) or fragment thereof or fusion protein, operably linked to a promoter. A variety of vectors and promoters can be used (e.g., a pCMV5 expression vectors).

The cell-based binding assays of the invention can be carried out by adding test compound (advantageously, bearing a detectable (e.g., radioactive or fluorescent) label) to medium in which the Tnni3k- (or fragment thereof or fusion protein containing same) expressing cells are cultured, incubating the test compound with the cells under conditions favorable to binding and then removing unbound test compound and determining the amount of test compound associated with the cells. As in the case of the cell-free assays, a test compound that binds to Tnni3k (or fragment thereof or fusion) is a candidate inhibitor of Tnni3k activity (e.g., kinase activity).

Cell-based assays can also take the form of competitive assays wherein a compound known to bind Tnni3k (and preferably labeled with a detectable label) is incubated with the Tnni3k- (or fragment thereof or fusion protein comprising same) expressing cells in the presence and absence of test compound. The affinity of a test compound for Tnni3k (or fragment or fusion) can be assessed by determining the amount of known compound associated with the cells incubated in the presence of the test compound, as compared to the amount associated with the cells in the absence of the test compound.

In a further embodiment, the present invention relates to a cell-based assay in which a cell that expresses Tnni3k or fragment thereof (or fusion protein comprising same) is contacted with a test compound and the ability of the test compound to inhibit Tnni3k activity is determined. The cell can be of mammalian origin, e.g., a cardiac cell (preferably, human). Determining the ability of the test compound to inhibit Tnni3k activity can be accomplished by monitoring, for example, Tnni3K autophosphorylation or Tnni3K phosphorylation of a cardiac specific protein or of MBP.

In a preferred embodiment, determining the ability of the test compound to inhibit the activity of Tnni3k can be effected by determining the ability of Tnni3k or fragment thereof (or fusion protein) to phosphorylate a target molecule (e.g., autophosphorylation of Tnni3K or phosphorylation of a cardiac specific protein or of MBP).

To determine the specific effect of any particular test compound (including a test compound selected on the basis of its ability to bind Tnni3k), assays can be conducted to determine the effect of various concentrations of the selected test compound on, for example, heart function.

The invention also includes the Tnni3k/CSQ transgenics described herein and methods of using same in screening compounds for therapeutic efficacy. The transgenics can be used to validate the in vivo efficacy of compounds selected as a result of in vitro screens. Efficacy can be determined by monitoring, for example, heart function (e.g., using echocardiography) or longevity.

In another embodiment, the invention relates to compounds identified using the above-described assays as being capable of binding to Tnni3k and/or inhibiting the effects of Tnni3k (e.g., kinase effects) on cellular bioactivities.

In a further embodiment of the invention, compounds that inhibit the activity (e.g., kinase activity) of Tnni3k can be administered to a mammal (human or non-human) to protect against, or reduce the risk of, heart failure, particularly when the mammal has cardiomyopathy. In accordance with this embodiment, the inhibitor can be administered in an amount sufficient to provide such protection or reduction in risk. It will be appreciated that the amount administered and dosage regime can vary, for example, with the inhibitor, the condition of the mammal and the effect sought. Based on the studies described in the Example that follows, it appears that inhibition of Tnni3k is effectively inconsequential for normal pathology. Thus, administration of inhibitors of Tnni3k activity can be expected to have minimal adverse side effects.

Tnni3k inhibitors identified in accordance with the above assays can be formulated as pharmaceutical compositions. Such compositions comprise the inhibitor and a pharmaceutically acceptable diluent or carrier. The inhibitor can be present in dosage unit form (e.g., as a tablet or capsule) or as a solution, preferably sterile, particularly when administration by injection is anticipated. As pointed out above, the dose and dosage regimen can vary, for example, with the patient, the compound and the effect sought. Optimum doses and regimens can be determined readily by one skilled in the art.

Techniques (e.g., siRNA or antisense stategies) that inhibit expression of Tnni3k also be used therapeutically to reduce the risk of heart failure.

Levels of Tnni3K can be used prognostically. Patients with elevated levels of Tnni3K can be expected to be at higher risk of heart failure.

In yet a further embodiment, the invention relates to kits, for example, kits suitable for conducting assays described herein. Such kits can include Tnni3k or fragment thereof, or fusion protein comprising same, e.g., bearing a detectable label. The kit can include an Tnni3k-specific antibody. The kit can further include ancillary reagents (e.g., buffers) for use in the assays. The kit can include any of the above components disposed within one or more container means.

Certain aspects of the invention are described in greater detail in the non-limiting Examples that follows. (See also U.S. Pat. Nos. 6,261,818, 6,500,654, 6,660,490, 6,987,000, 7,371,380, Feng et al, Biochemistry (Mosc.) 72:1199-204 (2007), Wang et al, J. Cell. Mol. Med., Nov. 16, 2007 (Epub ahead of print), Feng et al, Gen. Physiol. Biophys. 26:104-109 (2007), and Karaman et al, Nature Biotechnology 26:127-132 (2008)).

### EXAMPLE 1

### Experimental Details

Animal care and handling. All mice were handled according to approved protocol and animal welfare regulations of the Institutional Review Board at Duke University Medical Center. All inbred mouse strains used in the course of this study were obtained from Jackson Laboratory (Bar Harbor, Me.). Transgenic mice overexpressing CSQ (Jones et al, J. Clin. Invest. 101:1385-1393 (1998), Cho et al,

J. Biol. Chem. 274:22251-22256 (1999)) were maintained on a DBA/2J genetic background.

DBA.AKR-Hrtfm2 congenic mouse. Through repeated backcrossing to DBAJ2J, a congenic mouse was created harboring AKR genomic sequence at the Hrtfm2 locus in the DBA genetic background. At generation N2, breeders were selected which were heterozygous at Hrtfm2 and homozygous DBA at the other mapped modifier loci (Wheeler et al, Mamm. Genome 16:414-423 (2005)). Genome-wide SNP genotyping was carried out using the Mouse MD linkage panel with 1449 SNPs (Illumina, San Diego, Calif.). By generation N6, the animals were homozygous for DBA alleles throughout the genome and only showed heterozygosity for an approximately 10 Mb interval on chromosome 3, the region containing Hrtfm2. Once the generation N10 backcross had been reached, the DBA.AKR-Hrtfm2 mouse was maintained by intercross.

Mouse RNA isolation, microarray analysis and qRT-PCR. Whole hearts removed from age- and sex-matched wild type animals from each of the three primary strains (B6, DBA, AKR) were used to examine RNA transcript levels. Total RNA was isolated using the RNeasy Kit (Qiagen, Valencia, Calif.). Microarray analysis was done on an Affymetrix Mouse probe set (Mouse 430 2.0 Array, Affymetrix, Santa Clara, Calif.). Analysis was done using GeneSpring GX* 7.3 Expression Analysis (Agilent Technologies, Santa Clara, Calif.). For the TaqMan expression analysis, total RNA was extracted from whole mouse hearts using TRIzol reagent (Invitrogen, Carlsbad, Calif.). cDNA was synthesized from 1 μg total RNA using the High Capacity cDNA Archive Kit (Applied Biosystems, Foster City, Calif.) and used as the template for qRT-PCR. Tnni3k cDNA was amplified using the predesigned gene expression assay (TaqMan, ABI, assay ID: Mm01318633_ml). Beta-actin (Actb) was used as the endogenous control (TaqMan, ABI, catalogue number 4352341E). All amplifications were carried out in triplicate on an ABI Prism 7000 Real Time PCR system and analyzed with ABI software. All statistical analyses were done using an unpaired, two-tailed T-test.

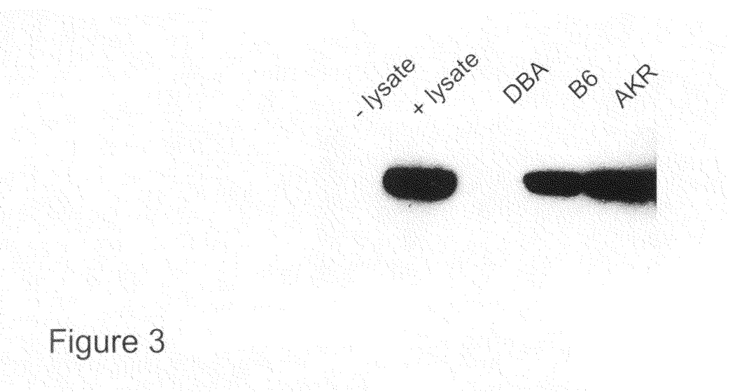

Analysis of Tnni3k protein expression. Whole heart protein lysates were prepared using flash-frozen heart tissue resuspended in lysis buffer with protease inhibitors. Lysates were analyzed by SDS-PAGE and Western blot performed with standard methods. A polyclonal peptide antiserum was developed to the C-terminal 14 amino acids of mouse Tnni3k protein (LHSRRNSGSFEDGN). Antiserum from 2 rabbits was purified on a Protein A column (GenScript, Piscataway, N.J.). Tnni3k antibody was used at a 1:1000 dilution in TBST with 5% dry milk. Protein bands can be visualized using secondary anti-rabbit antibody conjugated to HRP followed by incubation with Pierce SuperSignal West Pico Chemiluminescant Substrate (Thermo Fisher Scientific, Rockford, Ill.) and exposure to X-OMAT film (Kodak). Western blot analysis was used to confirm specificity of the antibody. As predicted, the mTnni3k antibody detects a 90 kDa protein from lysates prepared from 293T cells transiently transfected with a full length Tnni3k expression vector and in protein lysates from wild-type mouse hearts (FIG. 3).

Fluorescent RT-PCR assay. cDNAs were subjected to qRT-PCR using primers designed to detect either a 116 by or a 120 by cDNA PCR product. The forward primer was targeted 25 by upstream of the predicted 4 base insertion and was fluorescently labeled: 5′-6FAM-AGATTTCTGCAGTCCCTGGAT-3′ while the unlabeled reverse primer was targeted 48 by downstream of the predicted 4 base insertion with the sequence: 5′-AAGACATCAGCCTTGATGGTG-3′. Accumulation of both fragments was quantified using the GeneMapper analysis program on the ABI Prism 3730 DNA Sequencer (Applied Biosystems). Ratios of properly spliced and mis-spliced products were calculated based on relative amplification of both cDNA products.

Cloning of mTnni3k splicing constructs, cell culture and transfection. To create the Tnni3k genomic splicing constructs, DBA genomic DNA and B6 BAC clone RP23-180023 were used as templates to generate genomic 4 kb fragments that included part of intron 17, exon 18, intron 18, exon 19, intron 19, exon 20 and part of intron 20. The sequence of the forward PCR primer was 5% ACTTACTTATGTGCTTCTCTTAGTTATGTGC-3′; the reverse primer was 5′-GGATTTAAACATAGGTGTGTACCTAATT′GT-3′. PCR products were sub-cloned into pSPL3 (Invitrogen). Clones were verified by direct sequencing. Human embryonic kidney HEK293T (293T) cells (ATCC, Manassas, Va.) were maintained in Dulbecco's Modified Eagle's Medium (DMEM, Gibco) containing 10% fetal bovine serum at 37° C. in 5% CO₂. Cells were grown on 35 mm² plates and transfected with 1 μg plasmid DNA using FuGene reagent (Roche, Indianapolis, Ind.) according to the manufacturer's protocol. RNA was extracted with TRIzol (Invitrogen) 24 hr post-transfection and RT-PCR was carried out using standard methods.

In Vitro Splicing Assay. HEK293T cells were grown to approximately 80% confluence in 6-well plates, then transfected using with 1 μg of DBA- or B6-pSPL3 plasmid mixed with FuGene reagent. All transfections were performed in triplicate. Total RNA was extracted with TRIzol 20 hr post-transfection. RT-PCR was carried out using standard methods. Ratios of properly spliced and mis-spliced products for the Tnni3k construct were determined by the fluorescent RT-PCR assay described above.

Site-directed mutagenesis. A single base was changed at rs49812611 (IVS19+9), in the DBA-pSPL3 construct (G→A) and the B6-pSPL3 construct (A→G) using the QuikChange Site-Directed Mutagenesis Kit (Stratagene, LaJolla, Calif.) with PfuTurbo proofreading DNA polymerase. All clones were sequenced to verify proper incorporation of the SNP.

Culture of cardiomyocytes and NMD blocking experiments. HL-1 cardiomyocytes (Claycomb et al, Proc. Natl. Acad. Sci. USA 95:2979-2984 (1998)) were cultured in Claycomb Medium (SAFC Laboratories, Lenexa, Kans.) supplemented with Fetal Bovine Serum at 10%, 2 mM L-glutamine, 100 μg/ml Penicillin/Streptomycin, and 100 μM fungizone. Cells were cultured at 37° C. with 5% CO₂. Although the HL-1 cardiomyocytes were derived from a heart isolated from a mixed B6-DBA mouse (Claycomb et al, Proc. Natl. Acad. Sci. USA 95:2979-2984 (1998)), direct sequencing of genomic DNA from the cell line showed that it is homozygous for DBA alleles at the Tnni3k locus. HL-1 cells were treated with 5.7×10⁻² mM cycloheximide or 3.3×10⁻² mM emetine. Each treatment was performed in triplicate and RNA was isolated from cells 24 hours post treatment. RT-PCR was performed on RNA isolated from cells treated with NMD blocking drugs and untreated controls. Ratios of properly spliced and mis-spliced products were measured using the fluorescent RT-PCR splicing assay as described above. Total transcript levels were determined using the Tnni3k TaqMan assay described above.

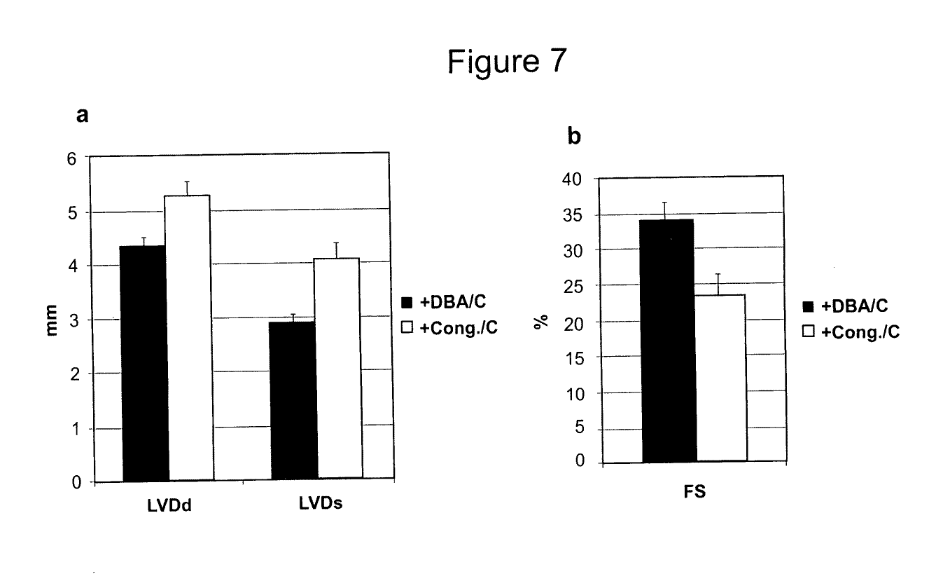

Creation and testing of a TNNI3K transgenic mouse. A full-length 2.5 kb TNNI3K cDNA was amplified from normal human heart RNA following RT-PCR and cloned into a vector downstream of the murine α-myosin heavy chain (αMHC) promoter. An artificial minx intron was inserted upstream of the TNNI3K start codon. The construct was linearized and an 8 kb fragment containing the αMHC promoter, cDNA and SV40 polyadenylation sequence was purified and used for microinjection. B6SJLF1/J blastocysts were injected with the linearized transgene and subsequently implanted into surrogate mice. The resulting founder animals were genotyped for presence of the TNNI3K transgene using a 5′ primer in the αMHC promoter and a 3′ primer in the TNNI3K transgene. Three transgenic lines were chosen for backcrossing to the DBA strain. Western blot analysis of heart lysates with a polyclonal antibody (Bethyl Laboratories, Montgomery, Tex.) raised against a human C-terminal TNNI3K peptide (FHSCRNSSSFEDSS) confirmed similar levels of expression of the TNNI3K transgene in each line (FIG. 7). This was repeated for several generations of backcrossing to DBA. Southern blot analysis of DNA from founder animals and subsequent generations (N2-N3) indicated that two founder lines carried 10-20 copies of the transgene while the third line appeared to have >100 copies. qRT-PCR with SYBRgreen (Invitrogen) was performed on heart cDNA from several transgenic mice to determine the relative expression difference between endogenous Tnni3k and transgenic TNNI3K expression.

M-mode echocardiography. Transthoracic two-dimensional M-mode echocardiography was performed between 12 and 18 weeks of age in conscious mice using either a Vevo 770 echocardiograph (Visual Sonics, Toronto, Canada) or an HDI 5000 echocardiograph with a 15-MHz frequency probe (Phillips Electronics, Bothell, Wash.). Measurements of cardiac function include heart rate, posterior and septal wall thickness, left-ventricular end diastolic diameter (LVEDD), left-ventricular end systolic diameter (LVESD) and ejection time (ET). Fractional shortening (FS) was calculated with the formula: FS=(LVEDD-LVESD)/LVEDD. The rate corrected mean velocity of fiber shortening (mVCFc) was calculated as previously described (Cho et al, J. Biol. Chem. 274:22251-22256 (1999)).

Transverse Aortic Constriction. Mice were anesthetized with a mixture of ketamine (100 mg/kg) and xylazine (2.5 mg/kg), and transverse aortic constriction (TAC) was performed as previously described (Rodman et al, Proc. Natl. Acad. Sci. USA 88:8277-8281 (1991)). TAC was performed on 14 TNNI3K transgene-positive animals and 14 transgene-negative (wild-type) littermates at 10 weeks of age. One of the transgene-negative controls and three transgene-positive animals died following surgery, which is a normal complication of this procedure. The remaining 24 mice were then analyzed by echocardiography (as described above), at 4 and 8 weeks following the surgery.

### Results

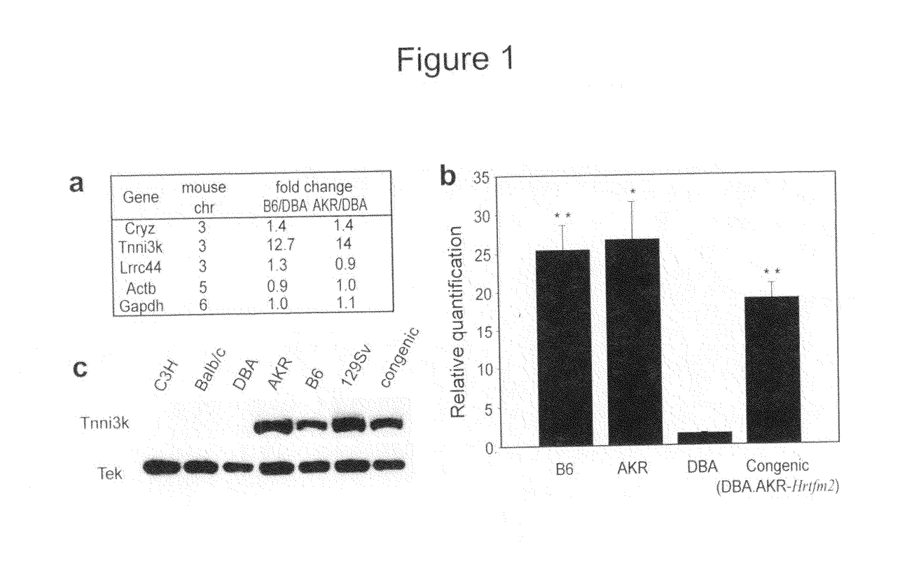

As part of an effort to identify candidate genes for the Hrtfm loci, microarray analysis of heart tissue from the strains used in these studies was performed to identify genes showing differences in transcript levels. Of the 21 genes mapping within the shared haplotype blocks (Wheeler et al, Mamm. Genome 16:414-423 (2005)) under the Hrtfm2 linkage peak, only one gene showed a greater than two-fold expression difference between the protected strain DBA/2J (DBA) and the susceptible strains C57/BL6 (B6) and AKR. Transcript levels of Tnni3k were 12-fold elevated in B6 and AKR compared to DBA, whereas the adjacent genes, as an example of all others within the interval, were not significantly elevated (FIG. 1A). These differences were validated by qRT-PCR showing 25-fold higher message levels in B6 and AKR strains, compared to DBA (FIG. 1B). In parallel to the genome-wide transcript level studies, the Hrtfm2 locus was genetically isolated by creating a congenic line that carries AKR alleles across Hrtfm2 and DBA alleles throughout the rest of the genome. Quantitative RT-PCR showed that Tnni3k transcript levels in hearts from DBA.AKR-Hrtfm2 congenic mice are comparable to levels observed in B6 and AKR (the source of the Hrtfm2 locus), and not that seen in DBA (the genomic background), suggesting that the Tnni3k expression differences are driven by cis-acting sequence elements at the Hrtfm2 locus, rather than trans-acting factors mapping elsewhere in the genome.

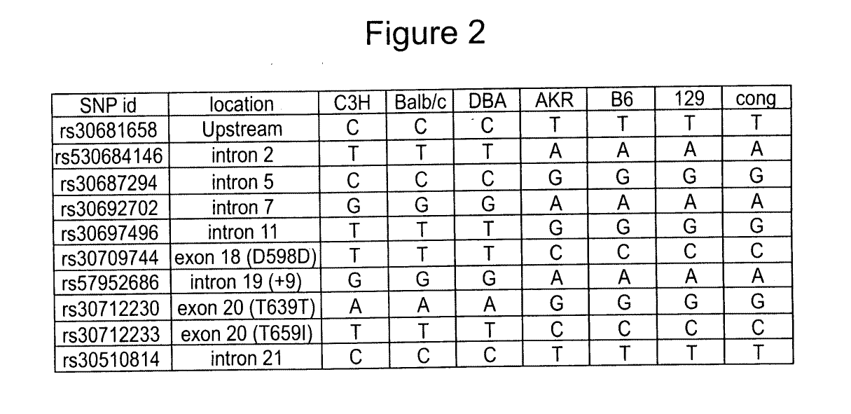

Heart tissue prepared from six inbred mouse strains was analyzed to determine if differences in levels of Tnni3k transcript are observed at the protein level. Three additional strains were chosen that share either the DBA or B6 haplotype at Tnni3k (FIG. 2). As predicted by transcript levels, robust levels of Tnni3k protein were detected in B6, AKR, 129X1/Sv and the DBA.AKR-Hrfm2 congenic, which share the B6 haplotype, but no protein was detected for DBA, A/J and Balb/c, which share the DBA haplotype (FIG. 1C). Thus, within the limits of detection of the antiserum (validated in FIG. 3), Tnni3k protein is absent from hearts of strains sharing the DBA haplotype across the gene. The latter strains effectively represent Tnni3k null genotypes with no apparent effect on development or survival, and with no obvious pathological consequence.

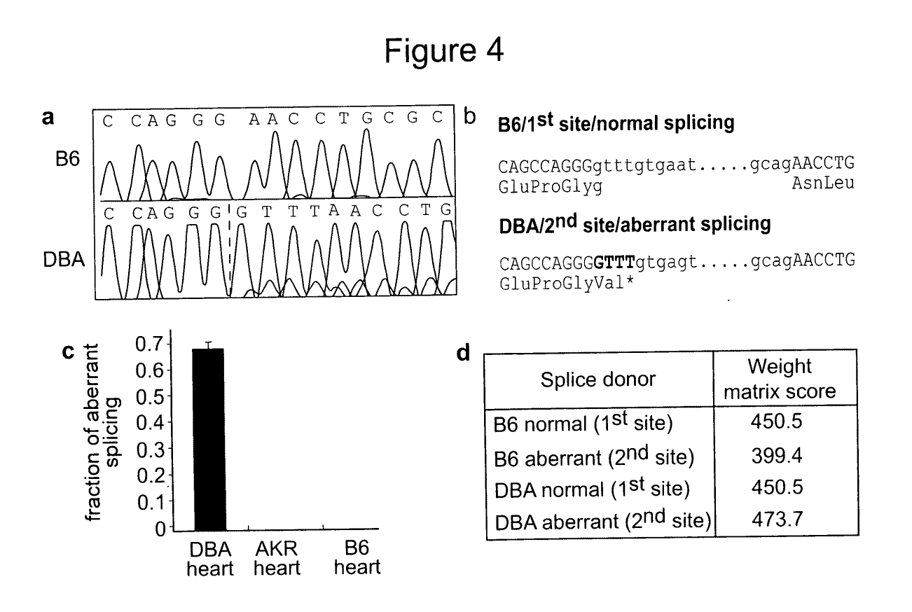

Tnni3k contains one non-synonymous and two synonymous SNPs (rs30712233, T659I; rs30709744, D598D; and rs30712230, T639T) between the relevant strains. By sequencing Tnni3k cDNAs, another strain-specific sequence alteration was noted. All strains with the B6 haplotype exhibit a major transcript identical to the published cDNA. In contrast, all strains with the DBA haplotype exhibit a mixture of two transcripts; the published transcript along with a second transcript containing a 4 nucleotide insertion between exons 19 and 20 (FIG. 4A). This insertion is not present in the genomic DNA, and represents the addition of 4 nucleotides from intron 19 into exon 19. The insertion creates a frameshift and an immediate premature termination codon (FIG. 4B). It was determined that the frameshifted transcript accounts for approximately 70% of the message in DBA heart mRNA but is not present in B6 or AKR (FIG. 4C). It is not found in any of the EST databases for mouse or for any other species, suggesting that it represents aberrant message created by defective splicing caused by the use of a second ‘gt’ splice donor site 4 nucleotides downstream of the normal donor site.

The genomic region surrounding exons 19 and 20 harbors over 50 SNPs. Although any of these could cause the aberrant splicing, focus was on the SNP nearest to the splice donor junction. B6 and related strains (AKR, 129X1/SvJ, MRL) show an ‘a’ at rs49812611, whereas DBA and related strains (A/J, C3H, Balb/c) show a ‘g’. This SNP lies at the +9 position for the normal splice site but at the +5 position for the aberrant splice site. Thus, DBA and related strains harbor the consensus ‘g’ sequence at the +5 position for the aberrant site. Weight matrix scores for splice donor strength (Staden, Nucleic Acids Res. 12:505-519 (1984), Burset et al, Nucleic Acids Res. 28:4364-4375 (2000)) for each possible splice donor site confirm that the second (aberrant) splice site is the strongest splice site in the region only when the ‘g’ nucleotide is present at rs49812611 (FIG. 4D).

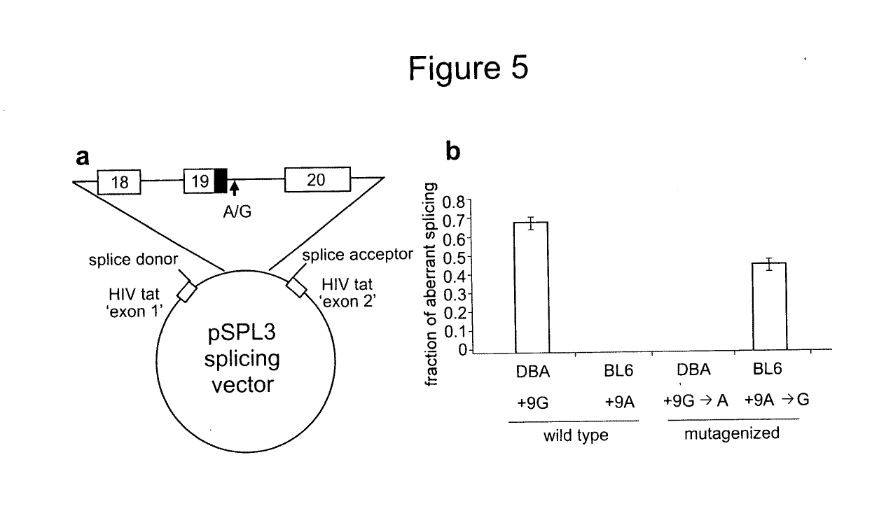

The hypothesis that rs49812611 is the cause of aberrant splicing was tested in an in vitro splicing system. Genomic DNA fragment spanning exons 18-20 from both B6 and DBA were sub-cloned and transfected into 293T cells. These in vitro constructs recapitulated the splicing pattern observed in vivo, confirming that the splicing defect is caused by cis-acting sequences residing on the cloned 4 kb fragment (FIG. 5B). Site-directed mutagenesis was used to investigate the role of rs49812611 in aberrant splicing. A single change at this SNP completely reverses the splicing pattern. DBA genomic DNA altered to carry the ‘a’ allele makes no aberrant splice product, whereas the B6 DNA carrying the ‘g’ allele does make the aberrant product (FIG. 5B). These results show that rs49812611 is responsible for the presence or absence of the aberrantly spliced message, although the full extent of aberrant splicing may be modulated by other sequence differences.

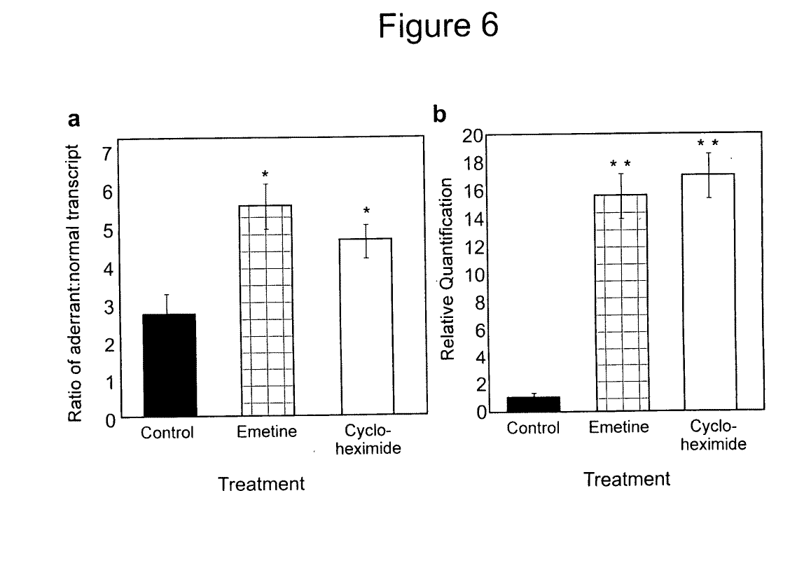

Since Tnni3k was originally identified as a positional candidate gene due to differences in transcript levels between strains, it was hypothesized that nonsense-mediated decay (NMD) is responsible for the drastically reduced levels of the frameshifted message in DBA. This was tested in the mouse cardiomyocyte cell line, HL-1 (Claycomb et al, Proc. Natl. Acad. Sci. USA 95:2979-2984 (1998)), which shares the DBA haplotype at Tnni3k. It was first confirmed that HL-1 cells express both aberrant and normal Tnni3k at levels comparable to wild-type DBA hearts, with the majority of the message including the 4 nucleotide insertion. HL-1 cardiomyocyte cells were then treated with two drugs that block NMD, cycloheximide and emetine (Carter et al, J. Biol. Chem. 270:28995-29003 (1995)). Treatment with either drug increased the level of aberrantly spliced transcript relative to the normally spliced message (FIG. 6A). As predicted, these treatments increased levels of total Tnni3k mRNA 16-fold (FIG. 6B), confirming that NMD plays a major role in the observed differences in transcript levels.

Although these experiments determined the molecular mechanism underlying the observed differences in Tnni3k transcript levels, they did not address the in vivo role of Tnni3k in the progression of cardiomyopathy. An investigation was next made as to whether Tnni3k was the gene underlying the Hrtfm2 locus. The Hrtfm2 congenic line (DBA.AKR-Hrtfm2) was first crossed to the CSQ transgenic sensitizer. This line retains the DBA genomic background for all chromosomes except chromosome 3, which contains approximately 10 Mb of the AKR genomic background encompassing Hrtfm2, including the AKR haplotype across the Tnni3k gene. The F1 animals resulting from this cross have only one copy of the AKR allele at Tnni3k, effectively reducing their expression level of Tnni3k in half relative to the parental AKR strain. The congenic line expressing one half a normal (AKR) dose of Tnni3k shows more dilated hearts and reduced heart function (decreased fractional shortening) relative to the DBA controls (FIG. 7). These data show that even Y2 a normal dose of Tnni3k results in accelerated dilation and cardiac malfunction in the context of heart disease.

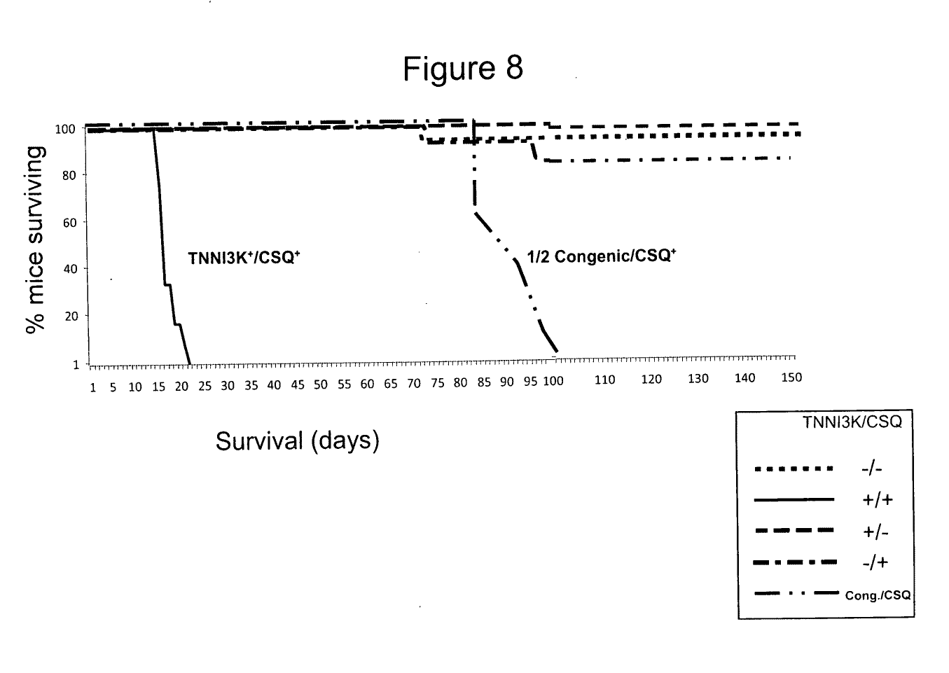

In the presence of the CSQ transgene, the DBA.AKR-Hrtfm2 congenic mice also show reduced survival in comparison to control mice. The congenic mice die by 100 days, showing that Y2 of the Tnni3k expression level seen in AKR causes decreased survival due to earlier onset of heart failure (FIG. 8).

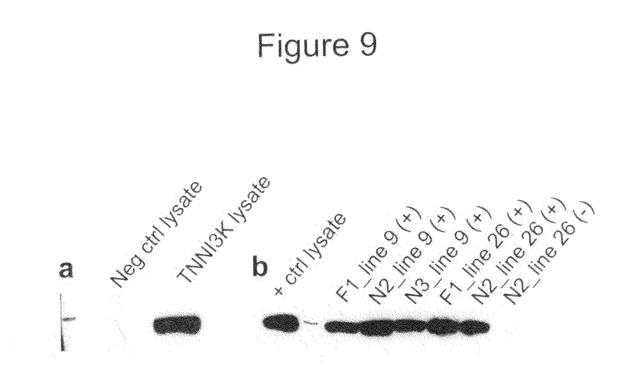

In order to validate that Tnni3k as responsible for this effect, three transgenic mouse lines were created that express human TNNI3K in the heart (FIG. 9). Quantitative RT-PCR showed that the human transgene is expressed at levels 5 to 20-fold above the endogenous B6 or AKR mouse transcript. The TNNI3K transgenes were introgressed into the DBA background (no detectable murine Tnni3k protein) to test the hypothesis that in the presence of the CSQ transgenic sensitizer, increased expression of TNNI3K would accelerate disease progression. F1 generation mice from all three lines survived over a year, and cardiac function in 12 and 21 week transgenic animals was indistinguishable from wild-type animals. Thus, TNNI3K expression alone does not result in overt cardiomyopathy or heart failure. This was not unexpected since in the absence of the CSQ transgene, there are no measurable differences in heart function between B6 and DBA animals, even though B6 express robust levels of Tnni3k whereas DBA shows no detectable protein.

By contrast, expression of TNNI3K in the context of the CSQ sensitizer results in severe cardiomyopathy leading to premature death (FIG. 8). Of the four possible genotypes from a cross between CSQ (sensitizer) and TNNI3K (modifier), only the double transgenics showed a dramatic decrease in survival. Whereas all other genotypes survived on average to at least 150 days (the experimental end point), animals expressing CSQ and TNNI3K died within 21 days. This premature death phenotype was similar to that previously observed when attempting to introgress the CSQ transgene into B6 (robust levels of endogenous Tnni3k). Starting with the sensitizer in the DBA background (Cho et al, J. Biol. Chem. 274:22251-22256 (1999)), it was not possible to move the CSQ transgene beyond the second generation, as N2 animals died at 30-40 days (Suzuki et al, Circulation 105:1824-1829 (2002).

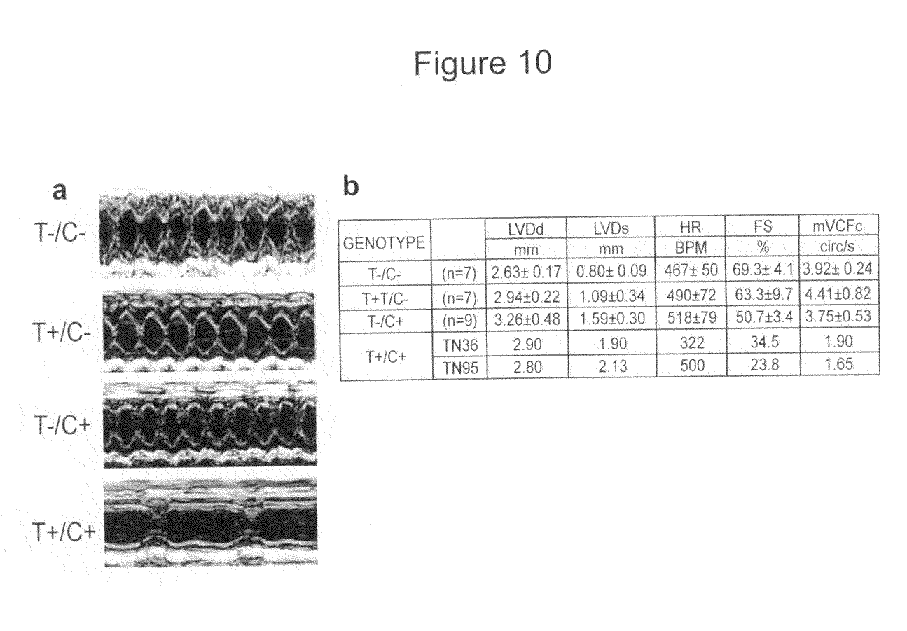

To determine whether the premature death was related to cardiac dysfunction, echocardiography was performed on animals with all four possible genotypes at 14 days, the earliest possible age for reproducible data. Only the double transgenic mice show abnormal heart function characterized by severe systolic dysfunction, chamber dilation, and decreased heart rate (FIG. 10). Due to severely impaired heart function and the risk of heart failure during the procedure, double transgenic animals used for survival measurements could not be used for parallel echocardiographic phenotyping. Three of five animals of this genotype died during echocardiography. Thus, the double transgenic animals develop cardiomyopathy by 14 days (or earlier) and die shortly after.

These data show that TNNI3K expression induces premature heart failure in the CSQ transgenic model of cardiomyopathy. An investigation was next made as to whether TNNI3K has a disease modifying effect in a model of cardiomyopathy unrelated to Calsequestrin over-expression. Transverse aortic constriction (TAC) induces left ventricular hypertrophy in response to pressure overload (Rodman et al, Proc. Natl. Acad. Sci. USA 88:8277-8281 (1991)). TAC-was performed on TNNI3K transgenic animals and wild-type littermate controls. Cardiac function was analyzed by echocardiography at 4 and 8 weeks following TAC surgery. The transgene-positive mice showed systolic dysfunction (increased LVEDs) and significantly reduced fractional shortening at 4 and 8 weeks post-surgery (FIG. 11). This confirms that TNNI3K overexpression has a detrimental effect on heart function outside the context of the CSQ sensitizer.

TNNI3K was identified as a cardiac-specific protein kinase that interacts with cardiac Troponin I (cTnI) (Zhao et al, J. Mol. Med. 81(5):297-304 (2003)). However, to date, cTnI has not been established as a phosphorylation target, and the in vivo function of TNNI3K remains uncertain. Regardless of the target of this novel kinase, it was shown that levels of TNNI3K are a major determinant of the rate of heart disease progression, since expression of this protein accelerates disease progression in two independent models of cardiomyopathy. Many inbred mouse strains are effectively null for this gene, but importantly, the null phenotype is protective. Drastically reduced levels of this protein, bordering on its absence, appear to have no effect on normal development or long-term survival, suggesting that inhibition of the kinase activity would have little or no pathological side-effects. Since protein kinases are critical cell cycle regulators, kinase inhibitors have become a major avenue for the development of novel cancer therapeutics. TNNI3K may be an ideal candidate for the development of similar small molecule kinase inhibitors in the context of heart disease. Null alleles of the Tnni3k orthologue would not be expected to exist in the human population, so that nearly all human cardiomyopathy patients would in principle be appropriate subjects for intervention at the level of kinase inhibition. Selective inhibition of TNNI3K would be particularly useful as it slows disease progression, and may prove beneficial in treating individuals with rapidly progressing heart disease. Further investigation of kinase inhibitors in the context of these disease models may lead to novel treatments for heart disease.

### EXAMPLE 2

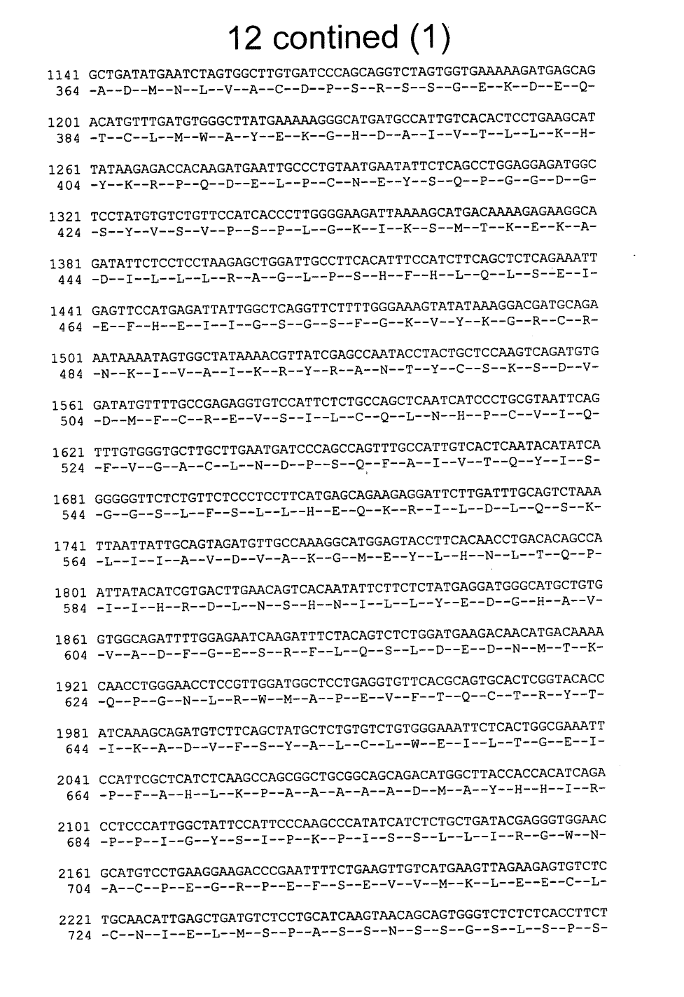

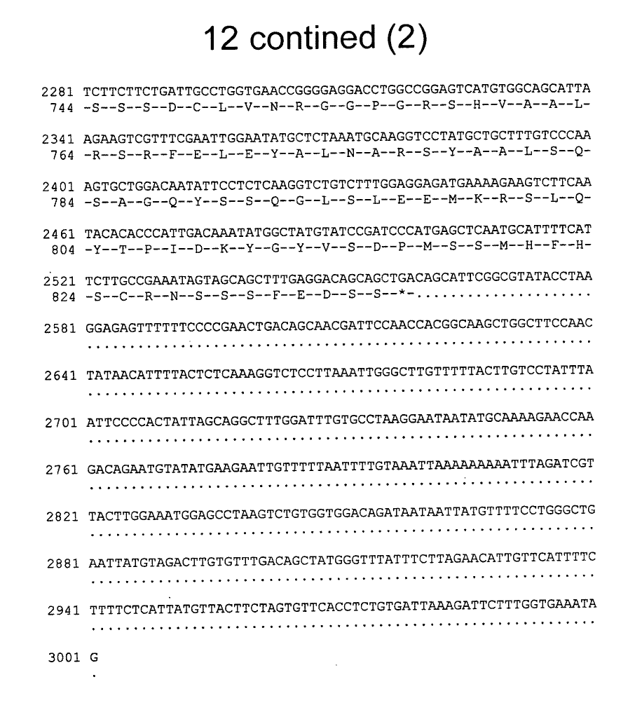

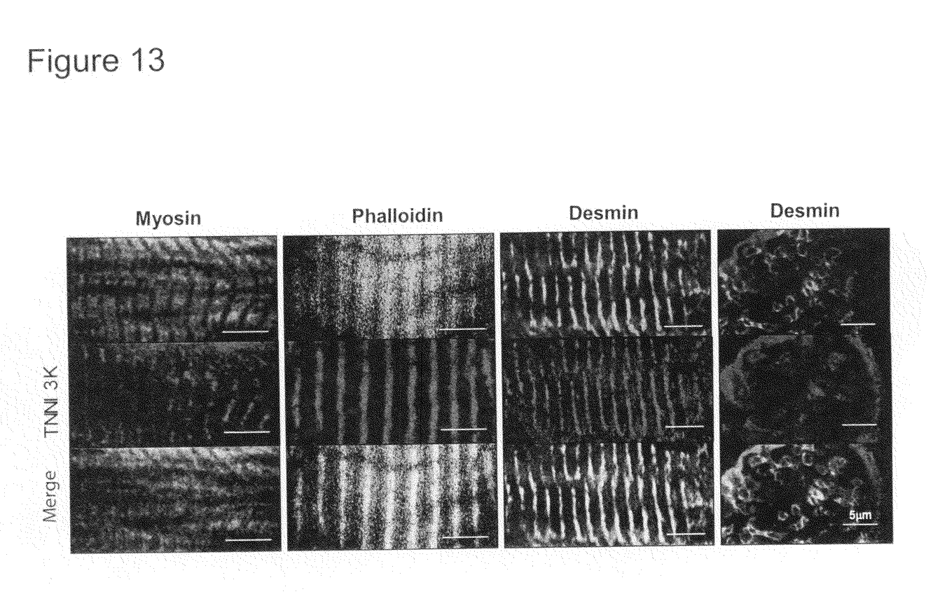

As a first step at determining the function of Tnni3k protein in the normal cardiomyocyte, its location within mouse heart tissue was investigated. Antiserum specific to human Tnni3k protein was used to probe the location of the exogenous (transgenic) protein in Tnni3k transgenic mice. These mice express the human Tnni3k protein from the heart-specific cardiac myosin heavy chain promoter. Importantly, these transgenic mice have been backcrossed into the DBA/2J background which express no detectable endogenous mouse Tnni3k protein. Thus, any staining is due to the human protein which is present in the mouse tissue. Tnni3k staining (red) shows a striated pattern of staining, consistent with it being a structural component of the cardiac sarcomere (FIG. 13). This is the first description of Tnni3k as a structural protein. The sarcomere is the primary structural unit of both cardiac and skeletal muscle and is directly responsible for muscle contraction.

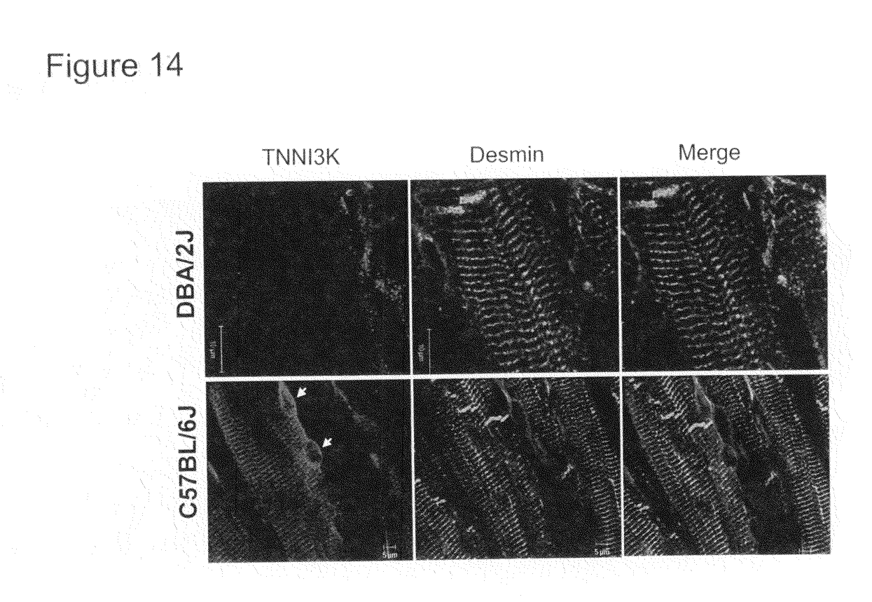

In order to determine where Tnni3k localizes within the complex sarcomere structure, the cardiac tissue sections were co-stained with antiserum to other proteins that are specific to the various components of the sarcomere. Tnni3k co-localizes only with desmin (yellow color in merged image), a classic marker of the Z-disk (also called the Z-line) of the sarcomere. The Z-disk is the site of attachment of critical components of the sarcomere, including the myosin and actin fᵢlaments. FIG. 14 shows that the normal mouse Tnni3k protein also shows the identical striated staining pattern and co-localizes with desmin. The location data of the human transgenic protein parallels that of the normal mouse protein showing that the transgenic data is not an artifact. Importantly, DBA/2J mice do no show this striated staining pattern, consistent with data that DBA/2J mice (and related strains) do not express this protein. This is the first description of Tnni3k as a sarcomere Z-disk protein. As shown in western blots, this protein is apparently completely dispensable, as DBA/2J and other strains with the same genetic haplotype at the mouse Tnni3k locus do not express any visible Tnni3k protein, and yet are completely normal in phenotype. Thus, Tnni3k provides a rational target for kinase inhibition, as it is dispensable and not required for normal heart function.

### EXAMPLE 3

### Experimental Details

Animal care and handling. All mice were handled according to approved protocols and animal welfare regulations of the Institutional Review Board at Duke University Medical Center. All inbred mouse strains used in the course of this study were obtained from Jackson Laboratory (Bar Harbor, Me.). Transgenic miceTNNI3K^{ig} were created as previously described (Wheeler et al; PLoS Genet. 5(9):e1000647 (2009)) and bred and maintained on a DBA/2J genetic background.

Cloning of TNNI3K constructs, cell culture and transfection. A full-length 2.5 kb human TNNI3K cDNA was amplified from normal human heart RNA following RT-PCR. Site-directed mutagenesis was used to change a single base in the hTNNI3K cDNA construct. The mutation, an ‘a’ to ‘g’, changed the AAA Lysine codon to an AGA Arginine codon at nucleotide position 1469/aa position 490. The truncated hTNNI3K isoforms were amplified from the full-length cDNA with specific primers. All hTNNI3K isoforms were cloned into pRK5 with a Flag tag at the amino terminus. FLAG-TNNI3K 5′:GGGAATTCATGGACTACAAG GACGAC GACGACCAAGGAAATTATAAATCTAGACC; FLAG-TNNI3K 3′: GGGAATT CCGCCGAATGCTGTCAGC; ANKR 3′: GCAAGCTTTGAGAG CTGAAGATG; KinaseDM 5′: GCGAATTCATGGACTACAAGGACGACGACGAC CAACATCTT CAGCTCTCA; SERT3′: GCAAGCTTCTGATGTCTCCTGCA; Human ACTC1 and myotilin cDNA were cloned into pRK5 with an HA tag at the amino terminus. HMYOT5′: GCGAATTCATGTACCCATACGACGTACCAGA TTACGCMT AACTACGAACGT; HMYOT3′: GC GAATTC TTA AAG TTC TTC ACT; HACTC1-5′: GCGAATTCGCCAAGATGTACCCATACGACGTACCAGATTA CGCTTGTGA CGACGAGGAGAC; HACTC1-3′: GCAAGCTTTTAGAAGCATT TGCGGTG.

Human embryonic kidney HEK293T (293T) cells (ATCC, Manassas, Va.) were maintained in Dulbecco's Modified Eagle's Medium (DMEM, Gibco) containing 10% fetal bovine serum at 37° C. in 5% CO₂. HL-1 cardiomyocytes were cultured in Claycomb Medium (SAFC Laboratories, Lenexa, Kans.) supplemented with Fetal Bovine Serum at 10%, 2 mM L-Glutamine, 100 mg/ml Penicillin/Streptomycin, and 100 mM fungizone. Cells were cultured at 37° C. with 5% CO₂. Cells were grown on 35 mm² plates and transfected with 1 μg plasmid DNA using FuGene reagent (Roche, Indianapolis, Ind.) or lipofectamine 2000 (Invitrogen) according to the manufacturer's protocol.

Immunoblotting and Immunoprecipitation. Whole heart protein lysates were prepared using flash-frozen heart tissue resuspended in lysis buffer with protease and phosphatase inhibitors. Lysates were analyzed by SDS-PAGE and western blotting was performed using standard methods. A polyclonal peptide antiserum (Bethyl Laboratories, Montgomery, Tex.) was raised against a mouse C-terminal TNNI3K peptide (LHSRRNSGSFEDGN). The antiserum was purified on a Protein A column (GenScript, Piscataway, N.J.), and was used at a 1:1000 dilution in TBST with 5% dry milk. Other primary antibodies were obtained from commercial sources; Mouse anti-Flag M2 (1:1000, sigma); Rabbit anti-HA (1:500, Sigma); mouse anti-alpha tubulin (1:500, DSHB, U. of Iowa). Protein bands were visualized using secondary antibodies conjugated to HRP (1:3000, BioRad) followed by incubation with Pierce SuperSignal West Pico Chemiluminescant Substrate (Thermo Fisher Scientific, Rockford, Ill.) and exposure to X-OMAT film (Kodak).

For immunoprecipitation, cell lysates were incubated with antibodies overnight at 4° C., then with protein A/G conjugated agarose beads (30 μl, Santa Cruz) for 2 hours. The pellet was washed three times with lysis buffer.

Immunocytochemistry. Cultured cells were fixed for 10 minutes in 4% paraformaldehyde/phosphate-buffered saline (PFA-PBS). Hearts were dissected and fixed overnight in 4% PFA-PBS. Fluorescent immunochemistry was performed on fixed cells or OCT embedded cryosections. Antisera used included Mouse anti-Flag (1:1000, sigma); Rabbit anti-HA (1:500; sigma); Rabbit anti-mTNNI3K (1: 50); Mouse anti-desmin (1:50, clone D33, DAKO); Mouse anti-myosin (1:50, DSHB, U. of Iowa). These were added to the blocking solution and were incubated by rocking at 4° C. overnight. Samples were rinsed three times for 30 min in PBT (PBS and 0.1% Triton X-100) with 5% BSA and 0.1% heat-inactivated goat serum, and incubated overnight at 4° C. in blocking solution with Alexa Fluor 594 phalloidin, Alexa Fluor 488 and 594 secondary antibodies (1:500; Invitrogen). Samples were washed three times for 30 min in PBT then mounted in ProLong Gold antifade reagent with DAPI (Invitrogen) and imaged on a Zeiss LSM420 confocal microscope or with a Coolsnap Pro digital camera (Roper Scientific, Trenton, N.J.) attached to an Olympus BX41 microscope.

### Results

TNNI3K associates with cardiac α-actin in diverse cell types. To determine the sub-cellular location of TNNI3K, transfected Flag-tagged human TNNI3K in COS-7 cells was immunostained. Flag-hTNNI3K distributes in the cytoplasm of COS-7 cells, and accumulates along cytoskeletal stress fibers (arrow head), suggesting that TNNI3K associates with actin filaments (FIG. 15A). The co-localization with actin filaments was further validated by co-transfection with HA-tagged human cardiac α-actin (hACTC-1), and by transfection in cardiomyocyte cell line HL-1 cell (FIG. 15A). HL-1 cells reflect an intracellular context more similar to primary cardiomyocytes, including the expression of sarcomere components (Claycomb et al, Proc. Natl. Acad. Sci. USA 95:62979-2984 (1998)). Furthermore, the physical interaction of TNNI3K and actin is supported by co-immunoprecipitation of Flag-hTNNI3K and HA-hACTC-1 from lysates of transfected cells (FIG. 15B).

TNNI3K localizes to the cardiac sarcomere at the Z disc. The interaction of TNNI3K and cardiac α-actin suggests that TNNI3K may be a sarcomeric protein, possibly localized to the thin filament of the sarcomere. Yeast two-hybrid analysis suggested that TNNI3K interacts with cardiac troponin I, which is also associated with the actin (thin) filament (Zhao et al, J. Mol. Med. 81(5):297-304 (2003)). To determine the intracellular localization of endogenous TNNI3K in cardiomyocytes from heart tissue, immunostaining was performed on cryosections of C57BL/6J and DBA/2J adult mouse hearts using an antibody directed against mouse C-terminal TNNI3K (FIG. 16A). In C57BL/6J mouse heart tissue, endogenous TNNI3K protein exhibits a striated pattern of expression, characteristic of a sarcomeric protein. TNNI3K also appears to accumulate around the nucleus of the cardiomyocytes (arrows in FIG. 16A), suggesting TNNI3K might anchor the nucleus to the sarcomere structures. It has been previously shown that TNNI3K protein is not expressed in heart lysates from DBA/2J mouse heart tissue (Wheeler et al, PLoS Genet. 5(9):e1000647 (2009)). Consistent with the western blot results, the characteristic striated localization pattern is not seen in heart tissue from DBA/2J mice. The striated Z disc expression pattern of TNNI3K protein suggested that the previously reported cardiac specific expression of the transcript may have been incorrect (Zhao et al, J. Mol. Med. 81(5):297-304 (2003)), and that TNNI3K may instead be an important component of all types of striated muscle. Thus, TNNI3K expression in skeletal muscle lysates was examined by western blot (FIG. 16B). However, no detectable expression was found in skeletal muscle from either inbred strain, suggesting that rather than playing a role in the common contractile function of the sarcomere, TNNI3K instead may specifically regulate cardiac contractility.

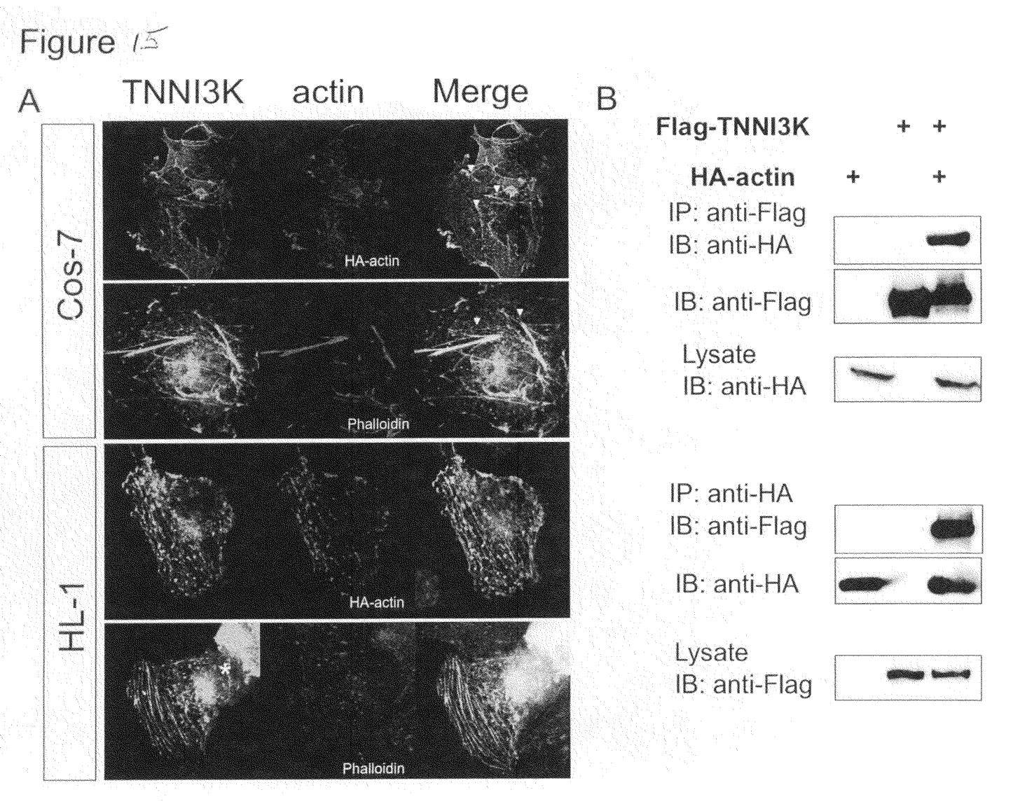

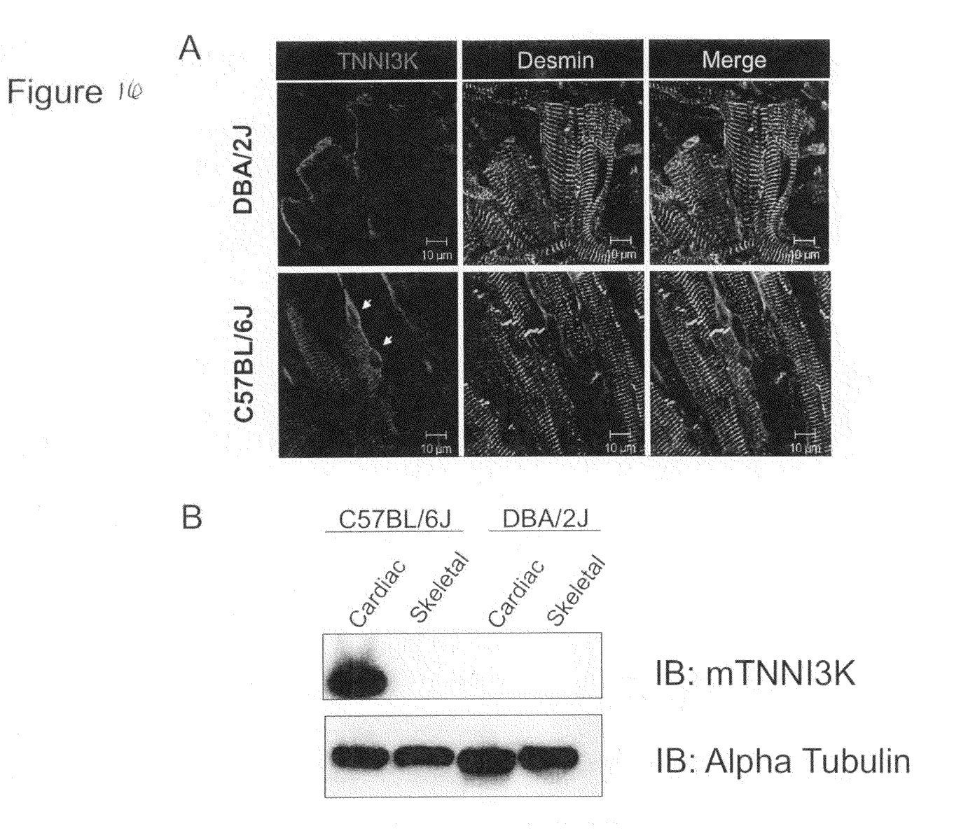

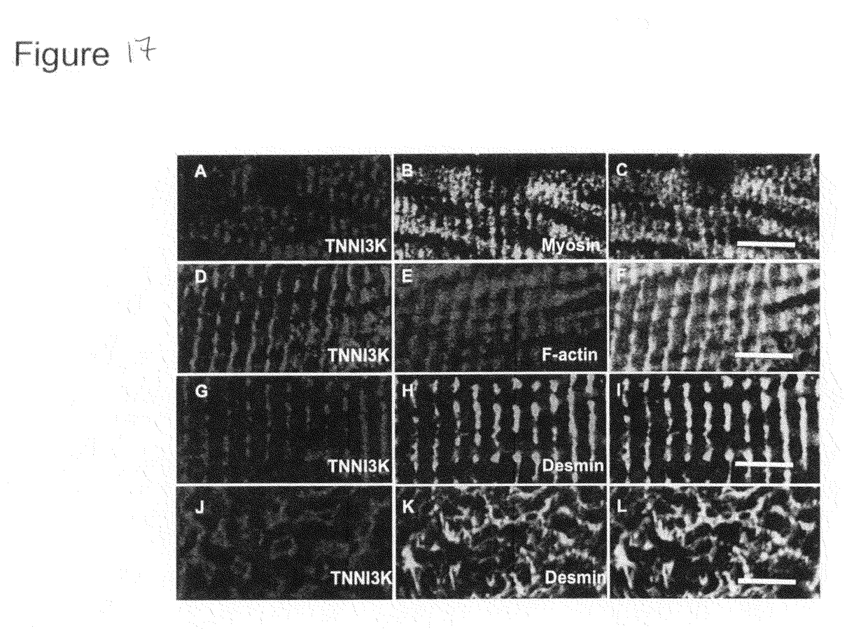

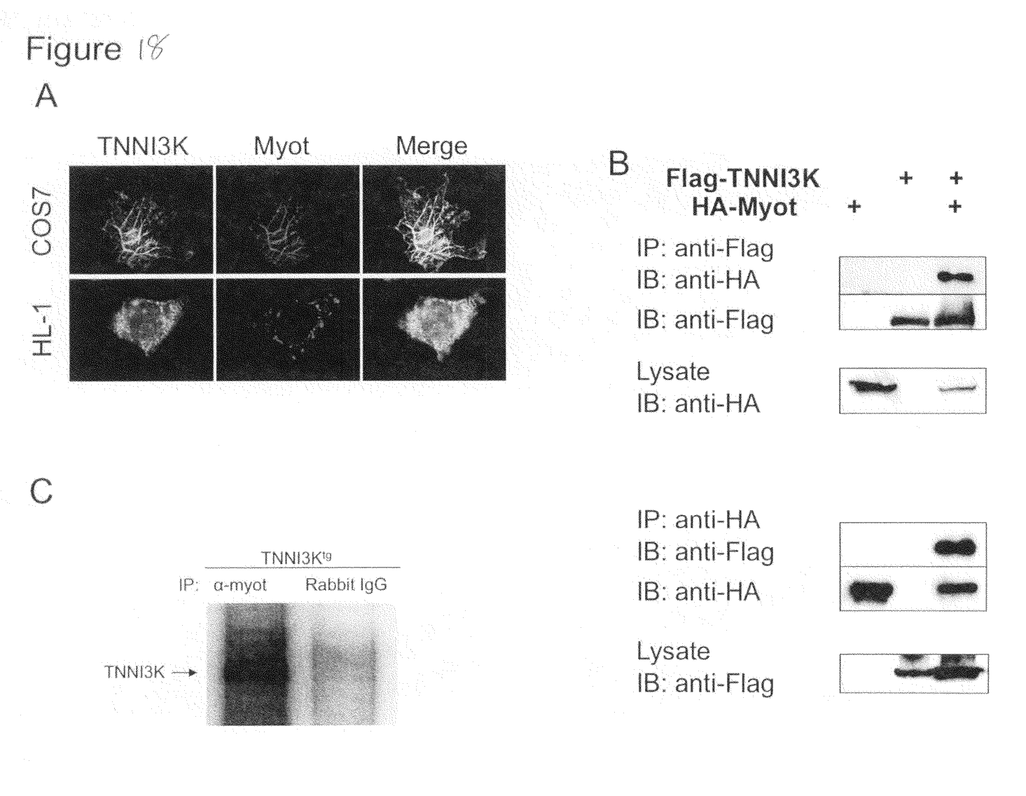

To determine the precise position of TNNI3K in the sarcomere, heart tissue was co-stained with markers for the various sarcomeric components (FIG. 17). TNNI3K shows a reciprocal (out-of-register) staining pattern with myosin that forms the sarcomere thick filaments, is centrally distributed along the actin thin filaments, and nearly perfectly overlaps with desmin, the intermediate filament protein surrounding the Z disc. In cross-section, TNNI3K localizes inside the desmin ring structures. Despite the apparent close proximity of TNNI3K and desmin in the sarcomere, it was not possible to co-immunoprecipitate TNNI3K and desmin from heart lysates (data not shown).

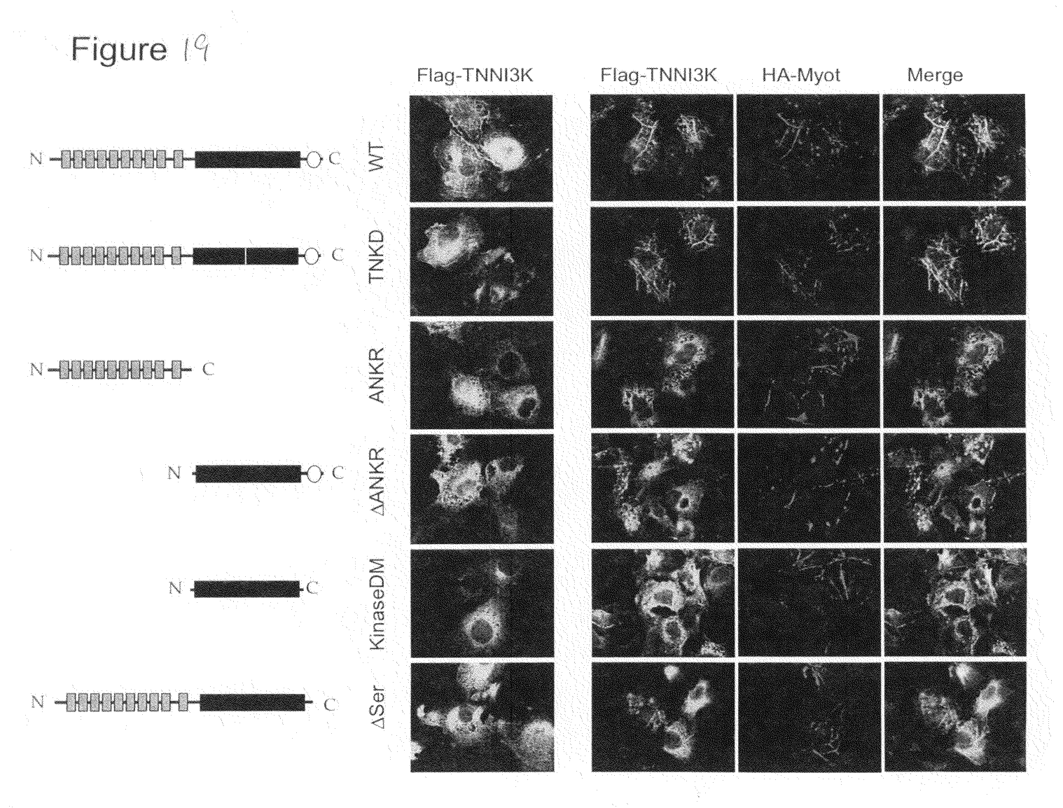

TNNI3K associates with the Z disc protein, myotilin. Although TNNI3K and actin can be immunoprecipitated together from heart lysates, its immunostaining pattern in heart tissue sections shows that TNNI3K does not localize across the entire actin (thin) filament. This suggests that rather than actin, another protein(s) anchors TNNI3K to the Z disc. Myotilin is an important scaffolding protein that associates with several important Z disc proteins (Salmikangas et al, Hum. Mol. Genet. 8:7 (1999), von Nandelstadh et al, Mol. Cell. Biol. 29(3):822-834 (2009)). Missense mutations in human myotilin cause the Mendelian disorder limb girdle muscular dystrophy 1A, where some patients display both skeletal muscle myopathy and cardiomyopathy (Hauser et al, Hum. Mol. Genet. 9(14):2141-2147 (2000)). Thus, an investigation was made of the relationship between TNNI3K and myotilin in different cellular contexts. Flag-TNNI3K and HA-myotilin co-localize in transfected COS-7 cells and HL-1 cells (FIG. 18A). Furthermore, HA-myotilin and Flag-TNNI3K can be co-immunoprecipitated from the lysates of transfected 293T cells (FIG. 18B), suggesting a physical interaction of these two proteins. Importantly, TNNI3K is co-immunoprecipitated with myotilin from mouse heart lysates (FIG. 18C), indicating that these two proteins exhibit strong binding in the appropriate tissue context. These data suggest that myotilin may be one of several proteins that anchor TNNI3K to the Z disc.

Functional mapping of TNNI3K domains. TNNI3K protein contains three recognizable domains/motifs: at the N-terminus, ten copies of an ankyrin repeat followed by the protein kinase domain and ending with a C-terminal Ser-rich domain. The Ser-rich domain in part appears to regulate kinase activity, since deletion of this domain increases TNNI3K autophosphorylation (Feng et al, Gen. Physiol. Biophys. 26(2):104-109 (2007)). To determine the domains of TNNI3K that are required for the actin/myotilin interaction, interaction domain mapping was performed by expressing various truncated versions of Flag-TNNI3K into COS-7 cells (FIG. 19). Full length TNNI3K revealed a characteristic cytoskeletal co-staining pattern. This pattern was not replicated by any of the individual domains alone. Deletion of serine-rich C-terminal tail did not completely abolish TNNI3K association with actin/myotilin filaments, but there was an increase of diffuse cytoplasm staining. These data suggest that the entire protein structure is required for complete TNNI3K interaction with actin and myotilin. Tight localization at the Z disc might require interaction with more than one protein, each interacting with a different domain of TNNI3K. An active kinase domain is not required for the association of TNNI3K and actin filaments in vitro (FIG. 19).

Thus, athough TNNI3K was first identified as a cardiac specific kinase in 2003 (Zhao et al, J. Mol. Med. 81(5):297-304 (2003)), and recent data from mouse models of cardiomyopathy demonstrate a pivotal role in disease progression, its biological function remains largely unknown. Here, certain of its cellular properties were determined as a first step in generating testable hypotheses regarding its function. TNNI3K associates with cytoskeletal actin in multiple cellular contexts. The actin thin filaments are a major component of the sarcomeric contractile apparatus, anchored at their plus end to the Z disc. In cardiac tissue, TNNI3K is localized precisely at the sarcomere Z disc.

The localization of TNNI3K at the Z disc suggests an important role in the regulation of cardiac contractility. The Z disc is the key interface between the contractile units and the cytoskeleton, by anchoring actin based thin filaments to titin from neighboring sarcomeres. Simultaneously, costameres (intermediate filaments and other proteins) circumscribe the Z disc and link the disc to the sarcolemma and the nucleus (Ervasti and Costameres, J. Biol. Chem. 278(16)L13591-13594 (2003)). It has been suggested that the Z disc is not only anchors the actin thin filaments but also “senses” mechanical stretch. Many new components of the Z disc participate in important signaling pathways (Knoll et al, Cell 111(7):943-955 (2002)). For example, muscle specific LIM protein (MLP) localizes at the Z disc and functions to sense stretch signals. MLP forms a complex with telethonin (T-cap) which caps the N-terminus of titin. Impairment of this complex uncouples the normal response to stretch signals (Knoll et al, Cell 111(7):943-955 (2002)). Regions of T-cap are rich in basic proteins and Ser/Thr residues, suggesting that the interaction of titin and T-cap might be regulated by phosphorylation (Mayans et al, Nature 395(6705):863-869 (1998)). However, the upstream kinase for T-cap has yet to be identified. Several other Z disc proteins are also critical for Z disc structure and for mediating stretch signaling, such as nexilin and calsarcins (Frank et al, J. Mol. Med. 84(6):446-468 (2006), Hassel et al, Nat. Med. 15(11):1281-1288 (2009), Frey et al, Nat. Med. 10(12):1336-1343 (2004)). TNNI3K phosphorylating its target at Z disc may likewise modulate the stretch signal response by phosphorylation of critical protein targets via action of its kinase domain.

The interaction of TNNI3K and the Z disc protein myotilin further supports the TNNI3K sub-cellular localization and also suggests potential targets of its kinase activity. In heart tissue, myotilin associates with many of the key components of the Z disc; α-actinin, filamin c (Salmikangas et al, Hum. Mol. Genet. 12(2):189-203 (2003)), the proteins of the FATZ family (calsarcin/myozenin) (Gontier et al, J. Cell. Sci. 118(Pt. 16):3739-3749 (2005)), and actin (Salmikangas et al, Hum. Mol. Genet. 12(2):189-203 (2003)). Myotilin bundles and stabilizes actin, and is thought to play a role in the organization and maintenance of Z disc integrity(Salmikangas et al, Hum. Mol. Genet. 12(2):189-203 (2003)). Phosphorylation of the PDZ binding motif of myotilin modulates the interaction with FATZ family members (von Nandelstradh et al, Mol. Cell Biol. 29(3):822-834 (2009)), suggesting that its phosphorylation status regulates its function. Furthermore, myotilin mutations are found in limb girdle muscular dystrophy 1A (Hauser et al, Hum. Mol. Genet. 9(14):2141-2147 (2000)). In addition to a skeletal muscle phenotype, some LGMD1A patients exhibit a dilated cardiomyopathy phenotype. Given its critical role at the Z disc, and its association with TNNI3K, myotilin may be a direct target of TNNI3K kinase activity. Conversely, it may instead serve as a platform to position TNNI3K near its appropriate phosphorylation target. These data provide indications as to TNNI3K function as a Z disc protein that regulates cytoplasmic signaling to sarcomeric structural proteins to modulate cardiac response to stress.

All documents and other information sources cited above are hereby incorporated in their entirety by reference.

What is claimed is:

1. A method of identifying a candidate inhibitor of cardiac Troponin I-interacting kinase (Tnni3k) activity comprising:
i) incubating Tnni3k, or fragment thereof, with a test compound, and
ii) assaying for binding of said test compound to said Tnni3k, or said fragment thereof,
wherein a test compound that binds to said Tnni3k, or said fragment thereof, is a candidate inhibitor of Tnni3k activity.

2. The method according to claim 1 wherein said fragment comprises a tyrosine kinase domain or an ankyrin repeat of Tnni3k.

3. The method according to claim 1 wherein, in step (i), a fusion protein comprising Tnni3k, or said fragment thereof, is incubated with said test compound.

4. The method according to claim 1 wherein said test compound is non-proteinaceous.

5. The method according to claim 1 wherein said test compound bears a detectable label.

6. The method according to claim 5 wherein said label is a radioactive or fluorescent label.

7. The method according to claim 1 wherein said Tnni3k, or said fragment thereof, is bound to a solid support.

8. The method according to claim 1 wherein said Tnni3k activity is kinase activity.

9. The method according to claim 1 wherein said method is a cell-free method.

10. The method according to claim 1 wherein said Tnni3k, or said fragment thereof, is present in a cell.

11. The method according to claim 10 wherein said cell is a cell expressing human Tnni3k.

12. The method according to claim 11 wherein said cell is a human cardiac cell.

13. The method according to claim 12 wherein said cardiac cell is a cardiomyocyte.

14. The method according to claim 11 wherein said cell is engineered to express human Tnni3k, or said fragment thereof.

15. The method according to claim 10 wherein said test compound is added to medium in which said cell is cultured.

16. A method of identifying a candidate inhibitor of Tnni3k activity comprising incubating Tnni3k, or fragment thereof, with a compound known to interact with Tnni3k and with a test compound, and determining the ability of said test compound to compete with said compound known to interact with Tnni3k for binding to said Tnni3k, or said fragment thereof,
wherein a test compound that competes with said compound known to interact with Tnni3 for binding to said Tnni3k, or said fragment thereof, is a candidate inhibitor of Tnni3k activity.

17. The method according to claim 16 wherein said compound known to interact with said Tnni3k is cardiac Troponin I (cTnI) or myelin basic protein (MBP).

18. The method according to claim 16 wherein said compound known to interact with said Tnni3k bears a detectable label.

19. A method of identifying an inhibitor of Tnni3k activity comprising culturing a cell that expresses Tnni3k, or fragment thereof having Tnni3k activity, in the presence and absence of a test compound and determining the ability of said Tnni3k, or said fragment thereof, to phosphorylate a target molecule in the presence and absence of said test compound, wherein a reduction in the level of phosphorylation of said target molecule in the presence of said test compound indicates said test compound is an inhibitor of Tnni3k activity.

20. The method according to claim 19 wherein said target molecule is Tnni3k, a cardiac specific protein or MBP.

21. A Tnni3k/CSQ transgenic animal.

22. A method of protecting against heart failure, or reducing the risk of heart failure, in a mammal in need thereof comprising administering to said mammal an amount of a compound that inhibits the activity of Tnni3k or the expression of Tnni3k sufficient to effect said protection or said reduction of risk.

23. The method according to claim 22 wherein said mammal has cardiomyopathy.

24. The method according to claim 22 wherein said method comprises administering an siRNA molecule or antisense molecule that inhibits expression of Tnni3k.

25. A candidate inhibitor of Tnni3k activity identifiable by the method of claim 1 or claim 16.

26. A composition comprising the candidate inhibitor of claim 25 and a pharmaceutically acceptable diluent or carrier.

27. An inhibitor of Tnni3k activity identifiable by the method of claim 19.

28. A composition comprising the inhibitor of claim 27 and a pharmaceutically acceptable diluent or carrier.


SEQUENCE LISTING (23 sequences)

SEQ ID NO: 1 - protein, 14 aa, Mus sp.
LHSRRNSGSFEDGN

SEQ ID NO: 2 - DNA, 21 bp, Artificial Sequence
  Description of Artificial Sequence Synthetic primer
agatttctgcagtccctggat

SEQ ID NO: 3 - DNA, 21 bp, Artificial Sequence
  Description of Artificial Sequence Synthetic primer
aagacatcagccttgatggtg

SEQ ID NO: 4 - DNA, 31 bp, Artificial Sequence
  Description of Artificial Sequence Synthetic primer
acttacttatgtgcttctcttagttatgtgc

SEQ ID NO: 5 - DNA, 30 bp, Artificial Sequence
  Description of Artificial Sequence Synthetic primer
ggatttaaacataggtgtgtacctaattgt

SEQ ID NO: 6 - protein, 14 aa, Homo sapiens
FHSCRNSSSFEDSS

SEQ ID NO: 7 - DNA, 55 bp, Artificial Sequence
  Description of Artificial Sequence Synthetic primer
gggaattcatggactacaaggacgacgacgaccaaggaaattataaatctagacc

SEQ ID NO: 8 - DNA, 25 bp, Artificial Sequence
  Description of Artificial Sequence Synthetic primer
gggaattccgccgaatgctgtcagc

SEQ ID NO: 9 - DNA, 23 bp, Artificial Sequence
  Description of Artificial Sequence Synthetic primer
gcaagctttgagagctgaagatg

SEQ ID NO: 10 - DNA, 50 bp, Artificial Sequence
  Description of Artificial Sequence Synthetic primer
gcgaattcatggactacaaggacgacgacgaccaacatcttcagctctca

SEQ ID NO: 11 - DNA, 23 bp, Artificial Sequence
  Description of Artificial Sequence Synthetic primer
gcaagcttctgatgtctcctgca

SEQ ID NO: 12 - DNA, 53 bp, Artificial Sequence
  Description of Artificial Sequence Synthetic primer
gcgaattcatgtacccatacgacgtaccagattacgcttttaactacgaacgt

SEQ ID NO: 13 - DNA, 23 bp, Artificial Sequence
  Description of Artificial Sequence Synthetic primer
gcgaattcttaaagttcttcact

SEQ ID NO: 14 - DNA, 61 bp, Artificial Sequence
  Description of Artificial Sequence Synthetic primer
gcgaattcgccaagatgtacccatacgacgtaccagattacgcttgtgacgacgaggagac

SEQ ID NO: 15 - DNA, 26 bp, Artificial Sequence
  Description of Artificial Sequence Synthetic primer
gcaagcttttagaagcatttgcggtg

SEQ ID NO: 16 - DNA, 15 bp, Mus sp.
ccagggaacctgcgc

SEQ ID NO: 17 - DNA, 16 bp, Mus sp.
ccaggggtttaacctg

SEQ ID NO: 18 - DNA, 19 bp, Mus sp.
cagccaggggtttgtgaat

SEQ ID NO: 19 - DNA, 10 bp, Mus sp.
gcagaacctg

SEQ ID NO: 20 - DNA, 19 bp, Mus sp.
  CDS (1)..(12)
cagccaggggtttgtgagt

SEQ ID NO: 21 - protein, 4 aa, Mus sp.
QPGV

SEQ ID NO: 22 - DNA, 3001 bp, Homo sapiens
  CDS (52)..(2556)
aatcttataacttgaagaactgccctggagaaaggaagaaacttataataaatgggaaattataaatctagaccaacccaaacttgtactgatgaatggaagaaaaaagtcagtgaatcatatgttatcacaatagaaagattagaagatgacctgcagatcaaggaaaaagaactgacagaactaaggaatatatttggctctgatgaagccttcagtaaagtcaatttaaattaccgcactgaaaatgggctgtctctacttcatttatgttgcatttgtggaggcaagaaatcacatattcgaactcttatgttgaaagggctccgcccatctcgactgacaagaaatggatttacagccttgcatttagcagtttacaaggataatgcagaattgatcacttctctgcttcacagtggagctgatatacagcaggttggatacggtggcctcactgccctccatattgctacaatagctggccacctagaggctgctgatgtgctgttgcaacatggagctaatgtcaatattcaagatgcagtttttttcactccattgcatattgcagcgtactatggacatgaacaggtaactcgccttcttttgaaatttggtgctgatgtaaatgtaagtggtgaagttggagatagacccctccacctagcatctgcaaaaggattcttgaatattgcaaaactcttgatggaagaaggcagcaaagcagatgtgaatgctcaagataatgaagaccatgtcccactccatttctgttctcgatttggacaccatgatatagttaagtatctgctgcaaagtgatttggaagttcaacctcatgttgttaatatctatggagatacccccttacacctggcatgctacaatggcaaatttgaagttgccaaggaaatcatccaaatatcaggaacagaaagtctgactaaggaaaacatcttcagtgaaacagcttttcatagtgcttgtacctatggcaagagcattgacctagtcaaatttcttcttgatcagaatgtcataaacatcaaccaccaaggaagggatgggcacactggattacactctgcttgctaccacggtcacattcgcctggttcagttcttactggataatggagctgatatgaatctagtggcttgtgatcccagcaggtctagtggtgaaaaagatgagcagacatgtttgatgtgggcttatgaaaaagggcatgatgccattgtcacactcctgaagcattataagagaccacaagatgaattgccctgtaatgaatattctcagcctggaggagatggctcctatgtgtctgttccatcacccttggggaagattaaaagcatgacaaaagagaaggcagatattctcctcctaagagctggattgccttcacatttccatcttcagctctcagaaattgagttccatgagattattggctcaggttcttttgggaaagtatataaaggacgatgcagaaataaaatagtggctataaaacgttatcgagccaatacctactgctccaagtcagatgtggatatgttttgccgagaggtgtccattctctgccagctcaatcatccctgcgtaattcagtttgtgggtgcttgcttgaatgatcccagccagtttgccattgtcactcaatacatatcagggggttctctgttctccctccttcatgagcagaagaggattcttgatttgcagtctaaattaattattgcagtagatgttgccaaaggcatggagtaccttcacaacctgacacagccaattatacatcgtgacttgaacagtcacaatattcttctctatgaggatgggcatgctgtggtggcagattttggagaatcaagatttctacagtctctggatgaagacaacatgacaaaacaacctgggaacctccgttggatggctcctgaggtgttcacgcagtgcactcggtacaccatcaaagcagatgtcttcagctatgctctgtgtctgtgggaaattctcactggcgaaattccattcgctcatctcaagccagcggctgcggcagcagacatggcttaccaccacatcagacctcccattggctattccattcccaagcccatatcatctctgctgatacgagggtggaacgcatgtcctgaaggaagacccgaattttctgaagttgtcatgaagttagaagagtgtctctgcaacattgagctgatgtctcctgcatcaagtaacagcagtgggtctctctcaccttcttcttcttctgattgcctggtgaaccggggaggacctggccggagtcatgtggcagcattaagaagtcgtttcgaattggaatatgctctaaatgcaaggtcctatgctgctttgtcccaaagtgctggacaatattcctctcaaggtctgtctttggaggagatgaaaagaagtcttcaatacacacccattgacaaatatggctatgtatccgatcccatgagctcaatgcattttcattcttgccgaaatagtagcagctttgaggacagcagctgacagcattcggcgtatacctaaggagagttttttccccgaactgacagcaacgattccaaccacggcaagctggcttccaactataacattttactctcaaaggtctccttaaattgggcttgtttttacttgtcctatttaattccccactattagcaggctttggatttgtgcctaaggaataatatgcaaaagaaccaagacagaatgtatatgaagaattgtttttaattttgtaaattaaaaaaaaatttagatcgttacttggaaatggagcctaagtctgtggtggacagataataattatgttttcctgggctgaattatgtagacttgtgtttgacagctatgggtttatttcttagaacattgttcattttcttttctcattatgttacttctagtgttcacctctgtgattaaagattctttggtgaaatag

SEQ ID NO: 23 - protein, 835 aa, Homo sapiens
MGNYKSRPTQTCTDEWKKKVSESYVITIERLEDDLQIKEKELTELRNIFGSDEAFSKVNLNYRTENGLSLLHLCCICGGKKSHIRTLMLKGLRPSRLTRNGFTALHLAVYKDNAELITSLLHSGADIQQVGYGGLTALHIATIAGHLEAADVLLQHGANVNIQDAVFFTPLHIAAYYGHEQVTRLLLKFGADVNVSGEVGDRPLHLASAKGFLNIAKLLMEEGSKADVNAQDNEDHVPLHFCSRFGHHDIVKYLLQSDLEVQPHVVNIYGDTPLHLACYNGKFEVAKEIIQISGTESLTKENIFSETAFHSACTYGKSIDLVKFLLDQNVININHQGRDGHTGLHSACYHGHIRLVQFLLDNGADMNLVACDPSRSSGEKDEQTCLMWAYEKGHDAIVTLLKHYKRPQDELPCNEYSQPGGDGSYVSVPSPLGKIKSMTKEKADILLLRAGLPSHFHLQLSEIEFHEIIGSGSFGKVYKGRCRNKIVAIKRYRANTYCSKSDVDMFCREVSILCQLNHPCVIQFVGACLNDPSQFAIVTQYISGGSLFSLLHEQKRILDLQSKLIIAVDVAKGMEYLHNLTQPIIHRDLNSHNILLYEDGHAVVADFGESRFLQSLDEDNMTKQPGNLRWMAPEVFTQCTRYTIKADVFSYALCLWEILTGEIPFAHLKPAAAAADMAYHHIRPPIGYSIPKPISSLLIRGWNACPEGRPEFSEVVMKLEECLCNIELMSPASSNSSGSLSPSSSSDCLVNRGGPGRSHVAALRSRFELEYALNARSYAALSQSAGQYSSQGLSLEEMKRSLQYTPIDKYGYVSDPMSSMHFHSCRNSSSFEDSS